SEQUENCE LISTING (1 sequence)

SEQ ID NO: 1 - protein, 406 aa, Human [sequence omitted]

(19) United States
(12) Patent Application Publication
(10) Pub. No.: US 2015/0307865 A1
(43) Pub. Date: Oct. 29, 2015

(54) COAGULATION FACTOR VII POLYPEPTIDES

(71) Applicant: Novo Nordisk Health Care AG, Zürich (CH)

(72) Inventors: Henning R. Stennicke, Kokkedal (DK); Ole Hvilsted Olsen, Broenshoej (DK); Henrik Oestergaard, Oelstykke (DK)

(21) Appl. No.: 14/675,887

(22) PCT Filed: Oct. 15, 2013
(86) PCT No.: PCT/EP2013/071510

§ 371 Date: Apr. 1, 2015

Related U.S. Application Data
(60) Provisional application No. 61/715,920, filed on Oct. 19, 2012.

(30) Foreign Application Priority Data
Oct. 15, 2012 (EP) 12188473.8

(51) Int. Cl. C12N 9/64

(52) U.S. Cl. CPC C12N 9/6437

(57) ABSTRACT

The present invention relates to modified coagulation Factor VII (Factor VII) polypeptides having coagulant activity as well as polynucleotide constructs encoding such polypeptides, vectors and host cells comprising and expressing such polynucleotides, pharmaceutical compositions, uses and methods of treatment.

### TECHNICAL FIELD

The present invention relates to modified coagulation factor VII (Factor VII) polypeptides having procoagulant activity. It also relates to polynucleotide constructs encoding such polypeptides, vectors and host cells comprising and expressing such polynucleotides, pharmaceutical compositions comprising such polypeptides, and uses and methods of treatment of such polypeptides.

### INCORPORATION-BY-REFERENCE OF THE SEQUENCE LISTING

SEQ ID NO. 1: Wild type human coagulation Factor VII

### BACKGROUND OF INVENTION

An injury to a blood vessel activates the haemostatic system that involves complex interactions between cellular and molecular components. The process that eventually causes the bleeding to stop is known as haemostasis. An important part of haemostasis is coagulation of the blood and the formation of a clot at the site of the injury. The coagulation process is highly dependent on the function of several protein molecules. These are known as coagulation factors. Some of the coagulation factors are proteases which can exist in an inactive zymogen or an enzymatically active form. The zymogen form can be converted to its enzymatically active form by specific cleavage of the polypeptide chain catalyzed by another proteolytically active coagulation factor. Factor VII is a vitamin K-dependent plasma protein synthesized in the liver and secreted into the blood as a single-chain glycoprotein. The Factor VII zymogen is converted into an activated form (Factor VIIa) by specific proteolytic cleavage at a single site, i.e. between R152 and 1153 of SEQ ID NO: 1, resulting in a two chain molecule linked by a single disulfide bond. The two polypeptide chains in Factor VIIa are referred to as light and heavy chain, corresponding to residues 1-152 and 153-406, respectively, of SEQ ID NO: 1 (wild type human coagulation Factor VII). Factor VII circulates predominantly as zymogen, but a minor fraction is on the activated form (Factor VIIa).

The blood coagulation process can be divided into three phases: initiation, amplification and propagation. The initiation and propagation phases contribute to the formation of thrombin, a coagulation factor with many important functions in haemostasis. The coagulation cascade starts if the single-layered barrier of endothelial cells that line the inner surface of blood vessels becomes damaged. This exposes subendothelial cells and extravascular matrix proteins to which platelets in the blood will stick to. If this happens, Tissue Factor (TF) which is present on the surface of sub-endothelial cells becomes exposed to Factor VIIa circulating in the blood. TF is a membrane-bound protein and serves as the receptor for Factor VIIa. Factor VIIa is an enzyme, a serine protease, with intrinsically low activity. However, when Factor VIIa is bound to TF, its activity increases greatly. Factor VIIa interaction with TF also localizes Factor VIIa on the phospholipid surface of the TF bearing cell and positions it optimally for activation of Factor X to Xa. When this happens, Factor Xa can combine with Factor Va to form the so-called “prothombinase” complex on the surface of the TF bearing cell. The prothrombinase complex then generates thrombin by cleavage of prothrombin. The pathway activated by exposing TF to circulating Factor VIIa and leading to the initial generation of thrombin is known as the TF pathway. The TF:Factor VIIa complex also catalyzes the activation of Factor IX to Factor IXa. Then activated Factor IXa can diffuse to the surface of platelets which are sticking to the site of the injury and have been activated. This allows Factor IXa to combine with FVIIIa to form the “tenase” complex on the surface of the activated platelet. This complex plays a key role in the propagation phase due to its remarkable efficiency in activating Factor X to Xa. The efficient tenase catalyzed generation of Factor Xa activity in turn leads to efficient cleavage of prothrombin to thrombin catalyzed by the prothrombinase complex.

If there are any deficiencies in either Factor IX or Factor VIII, it compromises the important tenase activity, and reduces the production of the thrombin which is necessary for coagulation. Thrombin formed initially by the TF pathway serves as a pro-coagulant signal that encourages recruitment, activation and aggregation of platelets at the injury site. This results in the formation of a loose primary plug of platelets. However, this primary plug of platelets is unstable and needs reinforcement to sustain haemostasis. Stabilization of the plug involves anchoring and entangling the platelets in a web of fibrin fibres.

The formation of a strong and stable clot is dependent on the generation of a robust burst of local thrombin activity. Thus, deficiencies in the processes leading to thrombin generation following a vessel injury can lead to bleeding disorders e.g. haemophilia A and B. People with haemophilia A and B lack functional Factor Villa or Factor IXa, respectively. Thrombin generation in the propagation phase is critically dependent on tenase activity, i.e. requires both Factor Villa and FIXa. Therefore, in people with haemophilia A or B proper consolidation of the primary platelet plug fails and bleeding continues.

Replacement therapy is the traditional treatment for hemophilia A and B, and involves intravenous administration of Factor VIII or Factor IX. In many cases, however, patients develop antibodies (also known as inhibitors) against the infused proteins, which reduce or negate the efficacy of the treatment. Recombinant Factor VIIa (Novoseven®) has been approved for the treatment of hemophilia A or B patients with inhibitors, and also is used to stop bleeding episodes or prevent bleeding associated with trauma and/or surgery. Recombinant Factor VIIa has also been approved for the treatment of patients with congenital Factor VII deficiency. It has been proposed that recombinant FVIIa operates through a TF-independent mechanism. According to this model, recombinant FVIIa is directed to the surface of the activated blood platelets by virtue of its Gla-domain where it then proteolytically activates Factor X to Xa thus by-passing the need for a functional tenase complex. The low enzymatic activity of FVIIa in the absence of TF as well as the low affinity of the Gla-domain for membranes could explain the need for supra-physiological levels of circulating FVIIa needed to achieve haemostasis in people with haemophilia.

Recombinant Factor VIIa has an in vivo functional half-life of 2-3 hours which may necessitate frequent administration to resolve bleedings in patients. Further, patients often only receive Factor VIIa therapy after a bleed has commenced, rather than as a precautionary measure, which often impinges upon their general quality of life. A recombinant Factor VIIa variant with a longer in vivo functional half-life would decrease the number of necessary administrations, support less frequent dosing and thus holds the promise of significantly improving Factor VIIa therapy to the benefit of patients and care-holders.

WO2007031559 (7012) discloses Factor VII variants with reduced susceptibility to inhibition by antithrombin.

WO2009126307 (Catalyst) discloses modified Factor VII polypeptides with altered procoagulant activity.

In general, there are many unmet medical needs in people with coagulopathies. The use of recombinant Factor VIIa to promote clot formation underlines its growing importance as a therapeutic agent. However, recombinant Factor VIIa therapy still leaves significant unmet medical needs, and there is a need for recombinant Factor VIIa polypeptides having improved pharmaceutical properties, for example increased in vivo functional half-life and improved activity.

### SUMMARY OF INVENTION

The present invention provides modified Factor VII polypeptides that are designed to have improved pharmaceutical properties. In a broad aspect, the invention relates to Factor VII polypeptides exhibiting increased in vivo functional half-life as compared to human wild-type Factor VIIa. In another broad aspect, the invention relates to Factor VII polypeptides exhibiting increased resistance to inactivation by endogenous plasma inhibitors, particularly antithrombin. In a further broad aspect, the invention relates to Factor VII polypeptides with enhanced or substantially preserved activity.

Provided herein are Factor VII polypeptides with increased in vivo functional half-life which comprise a combination of mutations conferring increased resistance to antithrombin inactivation and little or no loss of proteolytic activity. In a particularly interesting aspect of the present invention the Factor VII polypeptides are coupled to one or more “half-life extending groups” to increase the in vivo functional half-life.

In one aspect, the invention relates to a Factor VII(a) polypeptide comprising two or more substitutions relative to the amino acid sequence of human Factor VII (SEQ ID NO:1), wherein at least one of the substitutions is where T293 has been replaced by Lys (K), Tyr (Y), Arg (R) or Phe (F); where Q176 has been replaced by Lys (K), Arg (R), Asn (N); and/or Q286 has been replaced by Asn (N) and wherein at least one of the substitutions is where M298 has been replaced by Gln (Q), Lys (K), Arg (R), Asn (N). Gly (G), Pro (P), Ala (A), Val (V), Leu (L), Ile (I), Phe (F), Trp (W), Tyr (Y), Asp (D), Glu (E), His (H), Cys (C), Ser (S), or Thr (T).

In an interesting embodiment the invention relates to a Factor VII(a) polypeptide coupled with at least one half-life extending moiety.

In another aspect, the invention relates to a method for producing the Factor VII(a) polypeptide of the invention.

In a further aspect, the invention relates to a pharmaceutical composition comprising the Factor VII(a) polypeptide of the invention.

The general object of the present invention is to improve currently available treatment options in people with coagulopathies and to obtain Factor VII polypeptides with improved therapeutic utility. One object that the present invention has is to obtain Factor VII polypeptides with prolonged in vivo functional half-life while maintaining a pharmaceutically acceptable proteolytic activity. To achieve this, the Factor VII polypeptides of the present invention comprise a combination of mutations conferring reduced susceptibility to inactivation by the plasma inhibitor antithrombin while substantially preserving proteolytic activity; in particularly interesting embodiments of the present invention the Factor VII polypeptides are also coupled to one or more “half-life extending groups”.

Medical treatment with the modified Factor VII polypeptides of the present invention offers a number of advantages over the currently available treatment regimes, such as longer duration between injections, lower dosage, more convenient administration, and potentially improved haemostatic protection between injections.

### BRIEF DESCRIPTION OF DRAWINGS

FIG. 1 shows a model of the Factor VIIa/antithrombin (AT) complex. The model was generated by overlaying the protease domains of FVIIa (from the x-ray structure of the complex of FVIIa and TF, pdb entry: 1dan; Banner et al. 1996) and FXa (from the x-ray structure of the complex of FXa and AT, pdb entry: 2gd4; Johnson et al. 2006) by least square fitting procedure of the CA atoms and only retaining FVIIa and antithrombin. In the left orientation antithrombin (white, in cartoon representation) is placed on top of FVIIa (in solid surface representation) and in the right orientation a view into the active site (in black) and the substrate binding cleft of FVIIa is shown. The dark grey areas of the surface of FVIIa represent regions which have been explored for reduced antithrombin binding by point mutagenesis. In particular the amino acid residues Q176, Q286 and T293 (in black) have been explored by saturation mutagenesis.

FIG. 2 shows a sequence alignment (Higgins et al. 1992) of FVIIa heavy chains from a variety of species: human chimpanzee, dog, porcine, bovine, mouse rat, and rabbit. Upper and lower sequence numbering corresponds to chymotrypsin and FVII sequence numbering systems, respectively. The underlined residues are subject to mutagenesis.

FIG. 3 shows the pharmacokinetic profiles of FVIIa variants as semilogarithmic plots of clot activity (Clot) and FVIIa-antithrombin complex EIA (AT) levels after intravenous administration to Sprague Dawley rats. For several of the compounds, the concentration of FVIIa-antithrombin complex was below the detection limit of the assay at all or several of the time points as indicated by the lack of data points on the plot. Data are shown as mean±SD (n=3) with clot activity land FVIIa-antithrombin levels converted to nM. The graph title states the identity of the administered compound. Estimated pharmacokinetic parameters are given in Table 3

FIG. 4 shows the relationship between measured in vitro antithrombin reactivities and in vivo peak levels of FVIIa-antithrombin complexes. Peak levels of FVIIa-antithrombin complexes (denoted Cmax FVIIa-AT) were determined for a number of unmodified FVIIa variants following intravenous administration to Sprague Dawley rats as detailed in Example 12. The linear relationship confirms the predictiveness of the in vitro FVIIa variant screening procedure.

FIG. 5 shows the acute dose response of FVIIa Q176K and wild-type FVIIa dosed intravenously 5 min before transection of 4 mm of the tip of the tail in FVIII deficient mice (n=6/dose). Results are given as mean±SEM. The blood loss ED50 was calculated to 1.8 mg/kg for wild-type FVIIa and 2.6 mg/kg for FVIIa Q176K, respectively, p=0.50.

FIG. 6 shows the individual Cα-Cα distances from an LSQKAB superimposition calculation for the catalytic domains, the heavy chains, of 1) the FVIIa mutant Q176K and 2) that from the 1 DAN structure (Banner, D'Arcy, Chène, Winkler, Guha, Konigsberg, Nemerson, & Kirchhofer, 1996). The Cα-Cα distance for residue 176 is indicated with a dark spot.

FIG. 7 shows a theoretical model of the complex between antithrombin and FVIIa Q176K. In space filling are the relative positions of the two residues, Lys 176 of the FVIIa mutant Q176K and Arg 399 of antithrombin. The model was constructed from the structure of the antithrombin/FXa complex (Johnson, Li, Adams, & Huntington, 2006) where the FXa molecule has been superimposed by the heavy chain of FVIIa mutant Q176K molecule. The main chains of FVIIa, FXa and antithrombin are shown in ribbon representations. Residues Lys 176 of FVIIa and Arg 399 are labeled FVIIa-176K and ATM-399R, respectively.

### DESCRIPTION

The present invention relates to the design and use of Factor VII polypeptides exhibiting increased in vivo functional half-life, reduced susceptibility to inactivation by the plasma inhibitor antithrombin and preserved proteolytic activity. It has been found by the inventors of the present invention that specific combinations of mutations in human Factor VII in combination with conjugation to half-life extending moieties confer the above mentioned properties. The Factor VII polypeptides of the invention have an extended functional half-life in blood which is therapeutically useful in situations where a longer lasting pro-coagulant activity is wanted.

The present invention relates to a Factor VII(a) polypeptide comprising two or more substitutions relative to the amino acid sequence of human Factor VII (SEQ ID NO:1), wherein at least one of the substitutions is where T293 has been replaced by Lys (K), Tyr (Y), Arg (R) or Phe (F); where Q176 has been replaced by Lys (K), Arg (R), Asn (N); and/or Q286 has been replaced by Asn (N) and wherein at least one of the substitutions is where M298 has been replaced by Gln (Q), Lys (K), Arg (R), Asn (N). Gly (G), Pro (P), Ala (A), Val (V), Leu (L), Ile (I), Phe (F), Trp (W), Tyr (Y), Asp (D), Glu (E), His (H), Cys (C), Ser (S), or Thr (T).

Factor VII

Coagulation Factor VII (Factor VII) is a glycoprotein primarily produced in the liver. The mature protein consists of 406 amino acid residues defined by SEQ ID NO: 1 and is composed of four domains. There is an N-terminal gamma-carboxyglutamic acid (Gla) rich domain followed by two epidermal growth factor (EGF)-like domains and a C-terminal trypsin-like serine protease domain. Factor VII circulates in plasma predominantly as a single-chain molecule. Factor VII is activated to Factor VIIa by cleavage between residues Arg152 and Ile153, resulting in a two-chain protein held together by a disulphide bond. The light chain contains the Gla and EGF-like domains, while the heavy chain is the protease domain. Specific Glu (E) residues, i.e. E6, E7, E14, E16, E19, E20, E25, E26, E29 and E35, according to SEQ ID NO: 1 in Factor VII may be postranslationally gamma-carboxylated. The gamma-carboxyglutamic acid residues in the Gla domain are required for coordination of a number of calcium ions, which maintain the Gla domain in a conformation mediating interaction with phospholipid membranes.

The term “Factor VII(a)” encompasses the uncleaved single-chain zymogen, Factor VII, as well as the cleaved, two-chain and thus activated protease, Factor VIIa. “Factor VII(a)” includes natural allelic variants of Factor VII(a) that may exist and differ from one individual to another. A wild type human Factor VII sequence is provided in SEQ ID NO: 1.

Factor VII(a) may be plasma-derived or recombinantly produced, using well known methods of production and purification. The degree and location of glycosylation, gamma-carboxylation and other post-translational modifications may vary depending on the chosen host cell and its growth conditions.

Factor VII Polypeptides

The term “Factor VII(a) polypeptide” herein refers to wild type Factor VII(a) molecules as well as Factor VII(a) variants and Factor VII(a) conjugates. Such variants and conjugates may exhibit substantially the same, or improved, activity relative to wild-type human Factor VIIa.

The term “activity” of a Factor VII polypeptide, as used herein, refers to any activity exhibited by wild-type human Factor VII(a), and include, but is not limited to, coagulation or coagulant activity, pro-coagulant activity, proteolytic or catalytic activity such as to effect Factor X activation or Factor IX activation; ability to bind TF, Factor X or Factor IX; and/or ability to bind to phospholipids. These activities can be assessed in vitro or in vivo using recognized assays, for example, by measuring coagulation in vitro or in vivo. The results of such assays indicate that a polypeptide exhibits an activity that can be correlated to activity of the polypeptide in vivo, in which in vivo activity can be referred to as biological activity. Assays to determine activity of a Factor VII polypeptide are known to those of skill in the art. Exemplary assays to assess the activity of a FVII polypeptide include in vitro proteolysis assays, such as described in the Examples, below.

The term “increased or preserved activity”, as used herein, refers to Factor VIIa polypeptides that exhibit substantially the same or increased activity compared to wild type human Factor VIIa, for example i) substantially the same or increased proteolytic activity compared to recombinant wild type human Factor VIIa in the presence and/or absence of TF; ii) to Factor VII(a) polypeptides with substantially the same or increased TF affinity compared to recombinant wild type human Factor VIIa; iii) to Factor VII(a) polypeptides with substantially the same or increased affinity for the activated platelet; or iv) Factor VII(a) polypeptides with substantially the same or increased affinity/ability to bind to Factor X or Factor IX compared to recombinant wild type human Factor VIIa. For example preserved activity means that the amount of activity that is retained is or is about 10%, 20%, 30%, 40%, 50%, 60%, 70%, 80%, 90%, 100%, 200%, 300%, 400%, 500% or more of the activity compared to wild type human Factor VIIa.

The term “Factor VII(a) variant”, as used herein, is intended to designate a Factor VII having the sequence of SEQ ID NO: 1, wherein one or more amino acids of the parent protein have been substituted by another naturally occurring amino acid and/or wherein one or more amino acids of the parent protein have been deleted and/or wherein one or more amino acids have been inserted in the protein and/or wherein one or more amino acids have been added to the parent protein. Such addition can take place either at the N- or at the C-terminus of the parent protein or both. In one embodiment a variant is at least 95% identical with the sequence of SEQ ID NO: 1. In another embodiment a variant is at least 99% identical with the sequence of SEQ ID NO: 1. As used herein, any reference to a specific position refers to the corresponding position in SEQ ID NO: 1.

The terminology for amino acid substitutions used in this description is as follows. The first letter represents the amino acid naturally present at a position of SEQ ID NO:1. The following number represent the position in SEQ ID NO:1. The second letter represents the different amino acid substituting the natural amino acid. An example is K197A-Factor VII, wherein the Lysine at position 197 of SEQ ID NO:1 is replaced by a Alanine.

In the present context the three-letter or one-letter abbreviations of the amino acids have been used in their conventional meaning as indicated in table 1. Unless indicated explicitly, the amino acids mentioned herein are L-amino acids.

TABLE 1
Abbreviations for amino acids

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gin | Q |
| Asparagine | Asn | N |
| Glutamic Acid | Glu | E |
| Aspartic Acid | Asp | D |
| Serine | Ser | S |
| Threonine | Thr | T |

The term “Factor VII(a) conjugates” as used herein, is intended to designate a Factor VII polypeptide that exhibits substantially the same or improved biological activity relative to wild-type Factor VII(a), in which one or more of the amino acids or one or more of the attached glycan chains have been chemically and/or enzymatically modified, such as by alkylation, glycosylation, acylation, ester formation, disulfide bond formation, or amide formation.

The Factor VII polypeptides, of the invention, comprise two or more substitutions relative to the amino acid sequence of human Factor VII (SEQ ID NO:1),

wherein at least one of the substitutions is where T293 has been replaced by Lys (K), Tyr (Y), Arg (R) or Phe (F); where Q176 has been replaced by Lys (K), Arg (R), Asn (N); and/or Q286 has been replaced by Asn (N) and wherein at least one of the substitutions is where M298 has been replaced by Gln (Q), Lys (K), Arg (R), Asn (N). Gly (G), Pro (P), Ala (A), Val (V), Leu (L), Ile (I), Phe (F), Trp (W), Tyr (Y), Asp (D), Glu (E), His (H), Cys (C), Ser (S), or Thr (T).

In a series of interesting embodiments the invention relates to a Factor VII polypeptide, wherein the polypeptide has one of the following groups of substitutions T293K/M298Q, T293Y/M298Q, T293R/M298Q, T293F/M298Q, Q176K/M298Q, Q176R/M298Q, Q176N/M298Q, Q286N/M298Q, T293Y/V158D/E296V/M298Q, T293R/V158D/E296V/M298Q, T293K/V158D/E296V/M298Q, Q176K/V158D/E296V/M298Q and Q176R/V158D/E296V/M298Q.

### Half-Life-Resistance to Inactivation by Plasma Inhibitors

Besides in vivo clearance also in vivo functional half-life is of importance to the period of time during which the compound is “therapeutically available” in the body. The circulating half-life of recombinant human wild type Factor VIIa is about 2.3 hours (“Summary Basis for Approval for NovoSeven©”, FDA reference number 96 0597).

The term “in vivo functional half-life” is used in its normal meaning, i.e., the time required for reducing the biological activity of the Factor VII polypeptide remaining in the body/target organ with 50% in the terminal phase, or the time at which the activity of the Factor VII polypeptide is 50% of its initial value. Alternative terms to in vivo half-life include terminal half-life, plasma half-life, circulating half-life, circulatory half-life, and clearance half-life. The in vivo functional half-life may be deter-mined by any suitable method known in the art as further discussed below (Example 12).

The term “increased” as used about the in vivo functional half-life or plasma half-life is used to indicate that the relevant half-life of the polypeptide is increased relative to that of a reference molecule, such as wild-type human Factor VIIa as determined under comparable conditions.

In one aspect, the Factor VIIa polypeptides of the invention exhibit increased in vivo functional half-life relative to wild-type human Factor VIIa. For instance the relevant half-life may be increased by at least about 25%, such as by at least about 50%, e.g., by at least about 100%, 150%, 200%, 250%, or 500%.

Despite the detailed understanding of the biochemistry and pathophysiology of the coagulation cascade, the mechanistic basis for the clearance of the individual coagulation factors from circulation remains largely unknown. The marked differences in the circulating half-lives of Factor VII and its activated form Factor VIIa compared with zymogen and enzyme forms of other vitamin K-dependent proteins suggest the existence of specific and distinct clearance mechanisms for Factor VIIa. Two types of pathways appear to be operable in the clearance of Factor VIIa—one resulting in elimination of the intact protein, the other mediated by plasma inhibitors and leading to proteolytic inactivation.

Antithrombin III (antithrombin, AT) is an abundant plasma inhibitor and targets most proteases of the coagulation system including Factor VIIa. It is present at micromolar concentrations in plasma and belongs to the serpin family of serine protease inhibitors that irreversibly bind and inactivate target proteases by a suicide substrate mechanism. The inhibition by antithrombin appears to constitute the predominant clearance pathway of recombinant Factor VIIa in vivo following intravenous administration. In a recent study of the pharmacokinetics of recombinant Factor VIIa in haemophilia patients, about 60% of the total clearance could be attributed to this pathway (Agerso et al. (2011) J Thromb Haemost, 9, 333-338).

In some embodiments, the Factor VII(a) polypeptides of the invention exhibiting increased resistance to inactivation by the endogenous plasma inhibitors, particularly antithrombin, relative to wild-type human Factor VIIa.

In one embodiment of the invention, the Factor VIIa polypeptides exhibit an increased half-life due to resistance to inactivation by inhibitors, such as endogenous plasma inhibitors, such as antithrombin. Due to the higher resistance to inhibition of the Factor VII(a) polypeptide of the present invention compared to native Factor VIIa, a lower dose may be adequate to obtain a functionally adequate concentration at the site of action and thus it will be possible to administer a lower dose and/or with lower frequency to the subject having bleeding episodes or needing enhancement of the normal haemostatic system.

It has been found by the inventors of the present invention that Factor VII polypeptides with the following mutations T293Y, T293R, T293K, Q176K, Q176R, Q286N confer increased resistance to antithrombin inactivation. Without being bound by theory, this resistance to inhibitor inactivation, of these Factor VIIa polypeptide variants, may be achieved at the expense of diminished TF-independent activity, which may represent a shortcoming of these Factor VIIa polypeptide variants in terms of activity.

It is known that Factor VIIa polypeptides with the following mutations M298Q, and V158D/E296V/M298Q confer increased proteolytic activity. However, these Factor VIIa polypeptide variants also show increased susceptibility to inhibitor inactivation, which may represent a shortcoming of these Factor VIIa polypeptide variants in terms of in vivo functional half-life.

It has been found by the inventors of the present invention that by combining these two groups of mutations mentioned above an increased or preserved activity is achieved while maintaining high resistance to inhibitor inactivation. That is, the Factor VII polypeptides of the present invention comprising a combination of mutations exhibit increased resistance to antithrombin inactivation as well as substantially preserved proteolytic activity. When the Factor VII polypeptides of the invention are conjugated with one or more half-life extending moieties a surprisingly improved effect on half-life extension is achieved. Given these properties, such conjugated Factor VII polypeptides of the invention exhibit improved circulatory half-life while maintaining a pharmaceutically acceptable proteolytic activity.

### Additional Modifications

The Factor VII polypeptides of the invention may comprise further modifications, in particular further modifications which confer additional advantageous properties to the Factor VII polypeptide. Thus, in addition to at least two substitutions mentioned above, the Factor VII polypeptides of the invention may for example comprise further amino acid modification, e.g. one further amino acid substitution. In one such embodiment, the Factor VII polypeptide of the invention has an additional mutation or addition selected from the group R396C, Q250C, and 407C, as described e.g. in WO2002077218.

The Factor VII polypeptides of the invention may comprise additional modifications that are or are not in the primary sequence of the Factor VII polypeptide. Additional modifications include, but not limited to, the addition of a carbohydrate moiety, the addition of a half-life extending moiety, e.g. the addition of a, PEG moiety, an Fc domain, etc. For example, such additional modifications can be made to increase the stability or half-life of the Factor VII polypeptide.

### Half-Life Extending Moieties or Groups

The term “half-life extending moieties” and “half-life extending groups” are herein used interchangeably and understood to refer to one or more chemical groups attached to one or more amino acid site chain functionalities such as —SH, —OH, —COOH, —CONH2, —NH2, or one or more N- and/or O-glycan structures and that can increase in vivo circulatory half-life of proteins/peptides when conjugated to these proteins/peptides. Examples of half-life extending moieties include: Biocompatible fatty acids and derivatives thereof, Hydroxy Alkyl Starch (HAS) e.g. Hydroxy Ethyl Starch (HES), Poly Ethylen Glycol (PEG), Poly (Glyx-Sery)n (HAP), Hyaluronic acid (HA), Heparosan polymers (HEP), Phosphorylcholine-based polymers (PC polymer), Fleximers, Dextran, Poly-sialic acids (PSA), Fc domains, Transferrin, Albumin, Elastin like (ELP) peptides, XTEN polymers, PAS polymers, PA polymers, Albumin binding peptides, CTP peptides, FcRn binding peptides and any combination thereof.

In a particularly interesting embodiment, the Factor VII polypeptide of the invention is coupled with one or more protracting groups/half-life extending moieties.

In one embodiment Cysteine-conjugated Factor VII polypeptide of the invention have one or more hydrophobic half-life extending moieties conjugated to a sulfhydryl group of a cysteine introduced in the Factor VII polypeptide. It is furthermore possible to link protractive half-life extending moieties to other amino acid residues.

In one embodiment Factor VII polypeptide of the invention is disulfide linked to tissue factor, as described e.g. in WO2007115953.

In another embodiment Factor VII polypeptide of the invention is a Factor VIIa variant with increased platelet affinity.

### PEGylated Derivatives

“PEGylated Factor VII polypeptide variants/derivatives” according to the present invention may have one or more polyethylene glycol (PEG) molecules attached to any part of the FVII polypeptide including any amino acid residue or carbohydrate moiety of the Factor VII polypeptide. Chemical and/or enzymatic methods can be employed for conjugating PEG or other protractive groups to a glycan on the Factor VII polypeptide. An example of an enzymatic conjugation process is described e.g. in WO03031464. The glycan may be naturally occurring or it may be engineered in, e.g. by introduction of an N-glycosylation motif (NXT/S where X is any naturally occurring amino acid) in the amino acid sequence of Factor VII using methods well known in the art. “Cysteine-PEGylated Factor VII polypeptide variants” according to the present invention have one or more PEG molecules conjugated to a sulfhydryl group of a cysteine residue present or introduced in the FVII polypeptide.

### Heparosan Conjugates

Factor VII polypeptide heparosan conjugates according to the present invention may have one or more Heparosan polymer (HEP) molecules attached to any part of the FVII polypeptide including any amino acid residue or carbohydrate moiety of the Factor VII polypeptide. Chemical and/or enzymatic methods can be employed for conjugating HEP to a glycan on the Factor VII polypeptide. An example of an enzymatic conjugation process is described e.g. in WO03031464. The glycan may be naturally occurring or it may be engineered in, e.g. by introduction of an N-glycosylation motif (NXT/S where X is any naturally occurring amino acid) in the amino acid sequence of Factor VII using methods well known in the art.

“Cysteine-HEP Factor VII polypeptide conjugates” according to the present invention have one or more HEP molecules conjugated to a sulfhydryl group of a cysteine residue present or introduced in the FVII polypeptide.

In one interesting embodiment of the invention, the Factor VII polypeptide is coupled to a HEP polymer.

### Fusion Proteins

Fusion proteins are proteins created through the in-frame joining of two or more DNA sequences which originally encode separate proteins or peptides or fragments hereof. Translation of the fusion protein DNA sequence will result in a single protein sequence which may have functional properties derived from each of the original proteins or peptides. DNA sequences encoding fusion proteins may be created artificially by standard molecular biology methods such as overlapping PCR or DNA ligation and the assembly is performed excluding the stop codon in the first 5′-end DNA sequence while retaining the stop codon in the 3′end DNA sequence. The resulting fusion protein DNA sequence may be inserted into an appropriate expression vector that supports the heterologous fusion protein expression in a standard host organisms such as bacteria, yeast, fungus, insect cells or mammalian cells.

Fusion proteins may contain a linker or spacer peptide sequence that separate the protein or peptide parts which define the fusion protein. The linker or spacer peptide sequence may facilitate the correct folding of the individual protein or peptide parts and may make it more likely for the individual protein or peptide parts to retain their individual functional properties. Linker or spacer peptide sequences may be inserted into fusion protein DNA sequences during the in frame assembly of the individual DNA fragments that make up the complete fusion protein DNA sequence i.e. during overlapping PCR or DNA ligation.

### Fc Fusion Protein

The term “Fc fusion protein” is herein meant to encompass Factor VII polypeptides of this invention fused to an Fc domain that can be derived from any antibody isotype. An IgG Fc domain will often be preferred due to the relatively long circulatory half-life of IgG antibodies. The Fc domain may furthermore be modified in order to modulate certain effector functions such as e.g. complement binding and/or binding to certain Fc receptors. Fusion of FVII polypeptides with an Fc domain, which has the capacity to bind to FcRn receptors, will generally result in a prolonged circulatory half-life of the fusion protein compared to the half-life of the wt FVII polypeptides. Mutations in positions 234, 235 and 237 in an IgG Fc domain will generally result in reduced binding to the FcγRI receptor and possibly also the FcγRIIa and the FcγRIII receptors. These mutations do not alter binding to the FcRn receptor, which promotes a long circulatory half-life by an endocytic recycling pathway. Preferably, a modified IgG Fc domain of a fusion protein according to the invention comprises one or more of the following mutations that will result in decreased affinity to certain Fc receptors (L234A, L235E, and G237A) and in reduced C1q-mediated complement fixation (A330S and P331S), respectively. Alternatively, the Fc domain may be an IgG4 Fc domain, preferably comprising the S241P/S228P mutation.

Production of Factor VII Polypeptides

In one aspect, the invention relates to a method for producing Factor VII polypeptides. The Factor VII polypeptides described herein may be produced by means of recombinant nucleic acid techniques. In general, a cloned human wild-type Factor VII nucleic acid sequence is modified to encode the desired protein. This modified sequence is then inserted into an expression vector, which is in turn transformed or transfected into host cells. Higher eukaryotic cells, in particular cultured mammalian cells, are preferred as host cells.

In a further aspect, the invention relates to a transgenic animal containing and expressing the polynucleotide construct.

In a further aspect, the invention relates to a transgenic plant containing and expressing the polynucleotide construct.

The complete nucleotide and amino acid sequences for human wild-type Factor VII are known (see U.S. Pat. No. 4,784,950, where the cloning and expression of recombinant human Factor VII is described).

The amino acid sequence alterations may be accomplished by a variety of techniques. Modification of the nucleic acid sequence may be by site-specific mutagenesis. Techniques for site-specific mutagenesis are well known in the art and are described in, for example, Zoller and Smith (DNA 3:479-488, 1984) or “Splicing by extension overlap”, Horton et al., Gene 77, 1989, pp. 61-68. Thus, using the nucleotide and amino acid sequences of Factor VII, one may introduce the alteration(s) of choice. Likewise, procedures for preparing a DNA construct using polymerase chain reaction using specific primers are well known to persons skilled in the art (cf. PCR Protocols, 1990, Academic Press, San Diego, Calif., USA).

The nucleic acid construct encoding the Factor VII polypeptide of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd. Ed. Cold Spring Harbor Laboratory, Cold Spring Harbor, N.Y., 1989).

The nucleic acid construct encoding the Factor VII polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesised, e.g. in an automatic DNA synthesiser, purified, annealed, ligated and cloned in suitable vectors. The DNA sequences encoding the human Factor VII polypeptides may also be prepared by polymerase chain reaction using specific primers, for instance as described in U.S. Pat. No. 4,683,202, Saiki et al., Science 239 (1988), 487-491, or Sambrook et al., supra.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct is preferably a DNA construct. DNA sequences for use in producing Factor VII polypeptides according to the present invention will typically encode a pre-pro polypeptide at the amino-terminus of Factor VII to obtain proper posttranslational processing (e.g. gamma-carboxylation of glutamic acid residues) and secretion from the host cell. The pre-pro polypeptide may be that of Factor VII or another vitamin K-dependent plasma protein, such as Factor IX, Factor X, prothrombin, protein C or protein S. As will be appreciated by those skilled in the art, additional modifications can be made in the amino acid sequence of the Factor VII polypeptides where those modifications do not significantly impair the ability of the protein to act as a coagulant.

The DNA sequences encoding the human Factor VII polypeptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the human Factor VII polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

Expression vectors for use in expressing Factor VIIa polypeptide variants will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell.

Examples of suitable promoters for directing the transcription of the DNA encoding the human Factor VII polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable a-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the Factor VII polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. Expression vectors may also contain a set of RNA splice sites located downstream from the promoter and upstream from the insertion site for the Factor VII sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nucl. Acids Res. 9:3719-3730, 1981) or the polyadenylation signal from the human Factor VII gene or the bovine Factor VII gene. The expression vectors may also include a noncoding viral leader sequence, such as the adenovirus 2 tripartite leader, located between the promoter and the RNA splice sites; and enhancer sequences, such as the SV40 enhancer.

To direct the Factor VII polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the human Factor VII polypeptides in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed human Factor VII polypeptides into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the human Factor VII polypeptides. The function of the leader peptide is to allow the expressed peptide to be directed from the endoplasmic reticulum to the Golgi apparatus and further to a secretory vesicle for secretion into the culture medium (i.e. exportation of the human Factor VII polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the Factor VII polypeptides, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. If, on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the Factor VII polypeptides of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human Factor VII polypeptide of interest.

The host cell into which the DNA sequences encoding the Factor VII polypeptides is introduced may be any cell, which is capable of producing the posttranslational modified human Factor VII polypeptides and includes yeast, fungi and higher eucaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk− ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk− ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human Factor VII polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the Factor VII polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The Factor VII polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

Transgenic animal technology may be employed to produce the Factor VII polypeptides of the invention. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and biochemically well characterized. Furthermore, the major milk proteins are present in milk at high concentrations (typically from about 1 to 15 g/l).

From a commercial point of view, it is clearly preferable to use as the host a species that has a large milk yield. While smaller animals such as mice and rats can be used (and are preferred at the proof of principle stage), it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk (see, for example, WO 88/00239 for a comparison of factors influencing the choice of host species). It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489), beta-lactoglobulin, a-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as a ˜4.25 kbp DNA segment encompassing the 5′ flanking promoter and non-coding portion of the beta-lactoglobulin gene (see Whitelaw et al., Biochem. J. 286: 31-39 (1992)). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta-lactoglobulin gene, is preferred. One such region is a DNA segment that provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other embodiments, the region surrounding the initiation ATG of the variant Factor VII sequence is replaced with corresponding sequences from a milk specific protein gene. Such replacement provides a putative tissue-specific initiation environment to enhance expression. It is convenient to replace the entire variant Factor VII pre-pro and 5′ non-coding sequences with those of, for example, the BLG gene, although smaller regions may be replaced.

For expression of Factor VII polypeptides in transgenic animals, a DNA segment encoding variant Factor VII is operably linked to additional DNA segments required for its expression to produce expression units. Such additional segments include the above-mentioned promoter, as well as sequences that provide for termination of transcription and polyadenylation of mRNA. The expression units will further include a DNA segment encoding a secretory signal sequence operably linked to the segment encoding modified Factor VII. The secretory signal sequence may be a native Factor VII secretory signal sequence or may be that of another protein, such as a milk protein (see, for example, von Heijne, Nucl. Acids Res. 14: 4683-4690 (1986); and Meade et al., U.S. Pat. No. 4,873,316, which are incorporated herein by reference).

Construction of expression units for use in transgenic animals is conveniently carried out by inserting a variant Factor VII sequence into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of a Factor VII variant; thereby creating a gene fusion that includes the expression control sequences of the milk protein gene. In any event, cloning of the expression units in plasmids or other vectors facilitates the amplification of the variant Factor VII sequence. Amplification is conveniently carried out in bacterial (e.g. E. coli) host cells, thus the vectors will typically include an origin of replication and a selectable marker functional in bacterial host cells. The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop to term. Offspring carrying the introduced DNA in their germ line can pass the DNA on to their progeny in the normal, Mendelian fashion, allowing the development of transgenic herds. General procedures for producing transgenic animals are known in the art (see, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49: 113-120 (1992); U.S. Pat. No. 4,873,191; U.S. Pat. No. 4,873,316; WO 88/00239, WO 90/05188, WO 92/11757; and GB 87/00458). Techniques for introducing foreign DNA sequences into mammals and their germ cells were originally developed in the mouse (see, e.g., Gordon et al., Proc. Natl. Acad. Sci. USA 77: 7380-7384 (1980); Gordon and Ruddle, Science 214: 1244-1246 (1981); Palmiter and Brinster, Cell 41: 343-345 (1985); Brinster et al., Proc. Natl. Acad. Sci. USA 82: 4438-4442 (1985); and Hogan et al. (ibid.)). These techniques were subsequently adapted for use with larger animals, including livestock species (see, e.g., WO 88/00239, WO 90/05188, and WO 92/11757; and Simons et al., Bio/Technology 6: 179-183 (1988)). To summarise, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed.

Production in transgenic plants may also be employed. Expression may be generalised or directed to a particular organ, such as a tuber (see, Hiatt, Nature 344:469-479 (1990); Edelbaum et al., J. Interferon Res. 12:449-453 (1992); Sijmons et al., Bio/Technology 8:217-221 (1990); and EP 0 255 378).

### Purification

The Factor VII polypeptides of the invention are recovered from cell culture medium. The Factor VII polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). Preferably, they may be purified by affinity chromatography on an anti-Factor VII antibody column. The use of calcium-dependent monoclonal antibodies, as described by Wakabayashi et al., J. Biol. Chem. 261:11097-11108, (1986) and Thim et al., Biochemistry 27: 7785-7793, (1988), is particularly preferred. Additional purification may be achieved by conventional chemical purification means, such as high performance liquid chromatography. Other methods of purification, including barium citrate precipitation, are known in the art, and may be applied to the purification of the novel Factor VII polypeptides described herein (see, for example, Scopes, R., Protein Purification, Springer-Verlag, N.Y., 1982).

For therapeutic purposes it is preferred that the Factor VII polypeptides of the invention are substantially pure. Thus, in a preferred embodiment of the invention the Factor VII polypeptides of the invention is purified to at least about 90 to 95% homogeneity, preferably to at least about 98% homogeneity. Purity may be assessed by e.g. gel electrophoresis and amino-terminal amino acid sequencing.

The Factor VII polypeptide is cleaved at its activation site in order to convert it to its two-chain form. Activation may be carried out according to procedures known in the art, such as those disclosed by Osterud, et al., Biochemistry 11:2853-2857 (1972); Thomas, U.S. Pat. No. 4,456,591; Hedner and Kisiel, J. Clin. Invest. 71:1836-1841 (1983); or Kisiel and Fujikawa, Behring Inst. Mitt. 73:29-42 (1983). Alternatively, as described by Bjoern et al. (Research Disclosure, 269 September 1986, pp. 564-565), Factor VII may be activated by passing it through an ion-exchange chromatography column, such as Mono Q® (Pharmacia fine Chemicals) or the like. The resulting activated Factor VII variant may then be formulated and administered as described below.

Assays

The invention also provides suitable assays for selecting preferred Factor VII polypeptides according to the invention. These assays can be performed as a simple preliminary in vitro test.

The activity of the Factor VIIa polypeptides can also be measured using a physiological substrate such as factor X (“In Vitro Proteolysis Assay”) (see Example 5), suitably at a concentration of 5-1000 nM (such as 30-40 nM) nM, where the factor Xa generated is measured after the addition of a suitable chromogenic substrate (e.g. S-2765). In addition, the activity assay may be run at physiological temperature.

The ability of the Factor VIIa polypeptides to generate thrombin can also be measured in an assay comprising all relevant coagulation factors and inhibitors at physiological concentrations (minus factor VIII when mimicking hemophilia A conditions) and activated platelets (as described on p. 543 in Monroe et al. (1997) Brit. J. Haematol. 99, 542-547 which is hereby incorporated as reference). See example 8.

Pharmaceutical Compositions

In one aspect, the present invention relates to compositions and formulations comprising a Factor VII polypeptide of the invention. For example, the invention provides a pharmaceutical composition that comprises a Factor VII polypeptide of the invention, formulated together with a pharmaceutically acceptable carrier.

Accordingly, one object of the invention is to provide a pharmaceutical formulation comprising a Factor VII polypeptide which is present in a concentration from 0.25 mg/ml to 100 mg/ml, and wherein said formulation has a pH from 2.0 to 10.0. The formulation may further comprise one or more of a buffer system, a preservative, a tonicity agent, a chelating agent, a stabilizer, an antioxidant or a surfactant, as well as various combinations thereof. The use of preservatives, isotonic agents, chelating agents, stabilizers, antioxidant and surfactants in pharmaceutical compositions is well-known to the skilled person. Reference may be made to Remington: The Science and Practice of Pharmacy, 19th edition, 1995.

In one embodiment, the pharmaceutical formulation is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. In another embodiment, the pharmaceutical formulation is a freeze-dried formulation, to which the physician or the patient adds solvents and/or diluents prior to use.

In a further aspect, the pharmaceutical formulation comprises an aqueous solution of a Factor VII polypeptide, and a buffer, wherein the polypeptide is present in a concentration from 1 mg/ml or above, and wherein said formulation has a pH from about 2.0 to about 10.0.

A Factor VII polypeptide of the invention may be administered parenterally, such as intravenously, such as intramuscularly, such as subcutaneously. Alternatively, a FVII polypeptide of the invention may be administered via a non-parenteral route, such as perorally or topically. An polypeptide of the invention may be administered prophylactically. An polypeptide of the invention may be administered therapeutically (on demand).

Therapeutic Uses

In a broad aspect, a Factor VII polypeptide of the present invention or a pharmaceutical formulation comprising said polypeptide may be used as a medicament.

In one aspect, a Factor VII polypeptide of the present invention or a pharmaceutical formulation comprising said polypeptide may be used to treat a subject with a coagulopathy.

In another aspect, a Factor VII polypeptide of the present invention or a pharmaceutical formulation comprising said polypeptide may be used for the preparation of a medicament for the treatment of bleeding disorders or bleeding episodes or for the enhancement of the normal haemostatic system.

In a further aspect, a Factor VII polypeptide of the present invention or a pharmaceutical formulation comprising said polypeptide may be used for the treatment of haemophilia A, haemophilia B or haemophilia A or B with acquired inhibitors.

In another aspect, a Factor VII polypeptide of the present invention or a pharmaceutical formulation comprising said polypeptide may be used in a method for the treatment of bleeding disorders or bleeding episodes in a subject or for the enhancement of the normal haemostatic system, the method comprising administering a therapeutically or prophylactically effective amount of a Factor VII polypeptide of the present invention to a subject in need thereof.

The term “subject”, as used herein, includes any human patient, or non-human vertebrates.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

The term “coagulopathy”, as used herein, refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic and are identified by a person skilled in the art. Non-limiting examples of congenital hypocoagulopathies are haemophilia A, haemophilia B, Factor VII deficiency, Factor X deficiency, Factor XI deficiency, von Willebrand's disease and thrombocytopenias such as Glanzmann's thombasthenia and Bernard-Soulier syndrome. The clinical severity of haemophilia A or B is determined by the concentration of functional units of FIX/Factor VIII in the blood and is classified as mild, moderate, or severe. Severe haemophilia is defined by a clotting factor level of <0.01 U/ml corresponding to <1% of the normal level, while people with moderate and mild haemophilia have levels from 1-5% and >5%, respectively. Haemophilia A with “inhibitors” (that is, allo-antibodies against factor VIII) and haemophilia B with “inhibitors” (that is, allo-antibodies against factor IX) are non-limiting examples of coagulopathies that are partly congenital and partly acquired.

A non-limiting example of an acquired coagulopathy is serine protease deficiency caused by vitamin K deficiency; such vitamin K-deficiency may be caused by administration of a vitamin K antagonist, such as warfarin. Acquired coagulopathy may also occur following extensive trauma. In this case otherwise known as the “bloody vicious cycle”, it is characterised by haemodilution (dilutional thrombocytopaenia and dilution of clotting factors), hypothermia, consumption of clotting factors and metabolic derangements (acidosis). Fluid therapy and increased fibrinolysis may exacerbate this situation. Said haemorrhage may be from any part of the body.

A non-limiting example of an iatrogenic coagulopathy is an overdosage of anticoagulant medication—such as heparin, aspirin, warfarin and other platelet aggregation inhibitors—that may be prescribed to treat thromboembolic disease. A second, non-limiting example of iatrogenic coagulopathy is that which is induced by excessive and/or inappropriate fluid therapy, such as that which may be induced by a blood transfusion.

In one embodiment of the current invention, haemorrhage is associated with haemophilia A or B. In another embodiment, haemorrhage is associated with haemophilia A or B with acquired inhibitors. In another embodiment, haemorrhage is associated with thrombocytopenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intracranially, intraaurally or intraocularly. In another embodiment, haemorrhage is associated with anticoagulant therapy.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### EXAMPLES

### Proteins

Human plasma-derived Factor X (FX) and Factor Xa (FXa) were obtained from Enzyme Research Laboratories Inc. (South Bend, Ind.). Soluble tissue factor 1-219 (sTF) or 1-209 were prepared according to published procedures (Freskgard et al., 1996). Expression and purification of recombinant wild-type FVIIa was performed as described previously (Thim et al., 1988; Persson and Nielsen, 1996). Human plasma-derived antithrombin (Baxter) was repurified by heparin sepharose chromatography (GE Healthcare) according to published procedures (Olson et al., 1993).

### Example 1

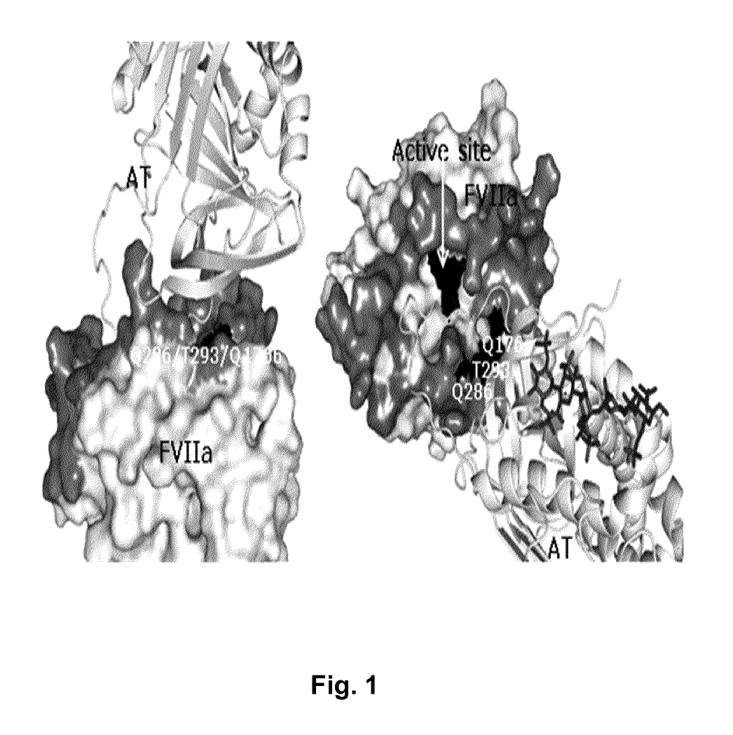

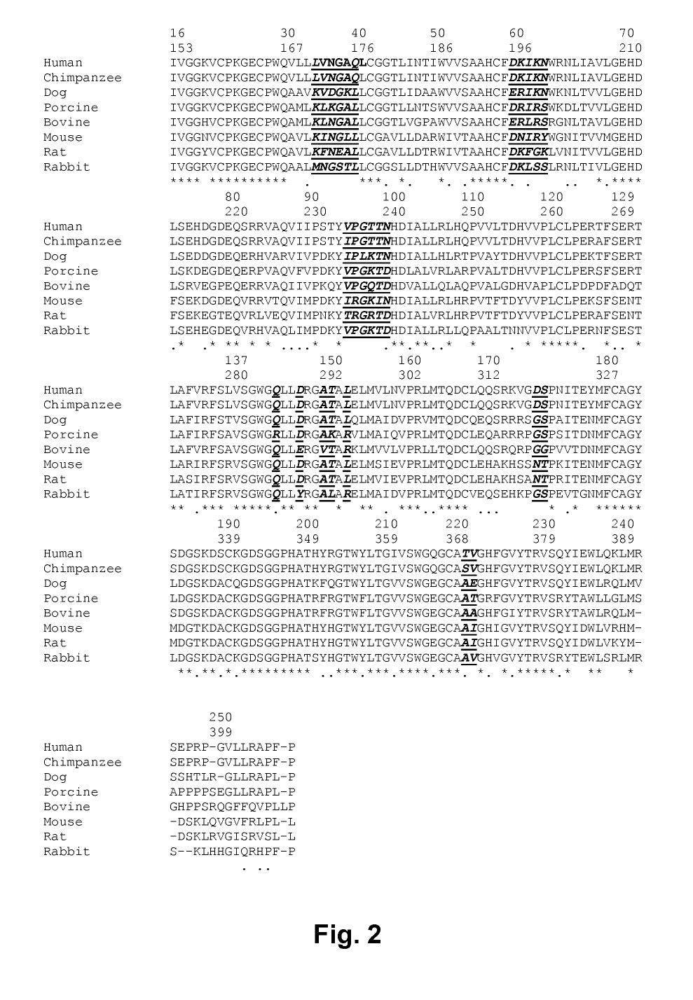

To map the FVIIa-antithrombin interaction, FVIIa variant libraries were designed in silico based on a structural model of the complex of FVIIa-antithrombin complex. The model shown in FIG. 1 was built using the published X-ray structure of FXa-antithrombin Michaelis complex as template (Johnson et al. 2006). FVIIa residues in close vicinity to antithrombin (the largest distance between FVIIa and antithrombin side chains was 12 Å) in the model were subject to mutagenesis. The first library was designed to explore the impact of conservative changes on human FVIIa binding to antithrombin. An alignment of FVII sequences from a variety of species (chimpanzee, dog, porcine, bovine, mouse, rat and rabbit) is shown in FIG. 2. A side chain in human FVIIa, in close vicinity to antithrombin, which in other species has a different side chain was mutated to that of the corresponding species. One example is the residue in position 286 being Gln in human FVII and Arg in porcine FVII. After screening the first library a second focused library was then designed where all or some of the possible amino acids substitutions (apart from Cys and Pro) in selected positions were tested. Examples include positions 176, 286 and 293 according to SEQ ID NO: 1.

### Example 2

Mutations were introduced into a mammalian expression vector encoding FVII cDNA using a site directed mutagenesis PCR-based method using KOD Xtreme™ Hot Start DNA Polymerase from Novagen or QuickChange® Site-Directed Mutagenesis kit from Stratagene. The following expression vectors were used: pTT5 (Durocher et al. (2002) Nucleic Acid Res. 30(2):e9) for transfection of HEK293F and HKB11 cells; pQMCF from Icosagen (Estonia) for transfection of CHOEBNALT85; pZEM219b (Busby et al. (1991) J. Biol. Chem., 266:15286-15292) and pMPSVHE (Artelt et al. (1988) Gene 62:213-219) for transfection of CHO-K1; pLN174 (Persson et al. (1996) FEBS Lett., 385:241-243) for transfection of BHK cells. Primers were designed according to manufacturer's recommendations. Introduction of the desired mutations was verified by DNA sequencing (MWG Biotech, Germany).

### Example 3

The FVII variants were expressed either in Baby Hamster Kidney (BHK) cells, Freestyle™ 293-F human embryonic Kidney cells (HEK293F; Gibco by Life Technologies, Naerum, DK), HKB11 (a hybrid cell line of HEK293 and a human B cell line) cells (ATCC, LGC Standards AB, Boras, Sweden), Chinese Hamster Ovarian (CHOK1) cells, or CHO-EBNALT85 cells from Icosagen Cell Factory, Estonia.

BHK adherent cells were transfected with FVII variant constructs using GeneJuice® from Merck Millipore (Hellerup, Denmark), according to manufacturer's instructions for production of stable cell lines. Methotrexate (Sigma-Aldrich) was used as selection reagent. Stable cell lines were cultured in medium to large scale giving a total of 5 to 10 litre cell supernatant. Cells were cultured in incubators at 37° C., 5 or 8% CO₂ in DMEM (Gibco by Life Technologies, Naerum, DK) supplemented with 2% (V/V) fetal calf serum (Gibco by Life Technologies, Naerum, DK), 1% (v/v) Pencillin/Steptomycin (Gibco by Life Technologies, Naerum, DK) and 5 mg/I Vitamin K₁ (Sigma-Aldrich).

HEK293F and HKB11 suspension cells were transient transfected using 293Fectin™ (Invitrogen by Life Technologies, Naerum, DK) according to manufacturer's instructions. Cells were cultured in shake incubators at 37° C., 5 or 8% CO₂ and 85 to 125 rpm. Transfected cells were expanded to a medium to large expression giving a total of 250 ml-1 litre cell supernatant. Supernatants were harvested by centrifugation followed by filtration through a 0.22 μM PES filter (Corning; Fischer Scientific Biotech, Slangerup, DK). HEK293F and HKB11 cells were cultured in Freestyle 293 Expression Medium (Gibco by Life Technologies, Naerum, DK) supplemented with 1% (v/v) Penicillin/Streptomycin (Gibco by LifeTechnologies, Naerum, DK) and vitamin K₁ (Sigma-Aldrich).

CHOEBNALT85 suspension cells were transient transfected by electroporation (Gene Pulse Xcell, Biorad, Copenhagen, DK). Transfected cells were selected with 700 μg/l Geneticin® (Gibco by Life Technologies), and expanded to medium/large giving a total of 500 ml to 10 litre supernatant. Cells were cultured in medium according to manufacturer's instructions supplemented with 5 mg/I Vitamin K₁ (Sigma-Aldrich). Cells were cultured in shake incubators at 37° C., 5 or 8% CO₂ and 85 or 125 rpm. Supernatants were harvested by centrifugation followed by filtration through a 0.22 μM PES filter (Corning; Fischer Scientific Biotech, Slangerup, DK).

CHOK1 cells adapted to suspension cells were transfected by electroporation (Gene

Pulse Xcell, Biorad, Copenhagen, DK) according to manufacturer's recommendations. 700 μg/l Geneticin® (Gibco by Life Technologies) were used as selection reagent. Stable cell lines were used for large-scale expressions. Cells were cultured in incubators at 37° C., 5 or 8% CO₂, and 85 or 125 rpm. Thermo Scientific Hyclone CDM4CHO™ medium supplemented with 1% (v/v) Penicillin/Streptomycin (Gibco by Life Technologies, Naerum, DK) and 5 mg/I Vitamin K₁ (Sigma-Aldrich) was used for expression. The supernatants were filtrated through a 0.22 μM PES filter (Corning, Fischer Scientific, Slangerup, DK).

Large-Scale Expression (BHK)—

Adherent BKH cell lines were cultivated in a DMEM/F12 medium (Invitrogen by Life Technologies, Naerum, DK) supplemented with 5 mg/I vitamin K1 and 2% fetal bovine serum (Invitrogen by Life Technologies, Naerum, DK). During propagation of seed culture for bioreactors, 10% fetal bovine serum was used and the medium was supplemented with 1 μM methotrexate (Sigma-Aldrich, Copenhagen, DK). Briefly, the cells were propagated in vented T-175 flasks, 2-layer and 10-layer cell factories incubated at 37° C. and 5% CO2. At confluency, cells were dissociated using TrypLE™ Express (Gibco by Life Technologies, Naerum, DK) prior to passaging to the next step. The production phase was performed as a repeated batch culture in bioreactors with microcarriers (5 g/L, Cytodex 3, GE Life Sciences, Uppsala, SE). pH was controlled around 7 by addition of CO2 and Na2CO3. Dissolved oxygen concentration was kept above 50% of saturation in air by sparging with oxygen. Temperature was maintained at 36.5° C. Withdrawn harvest was clarified through filtration (3 μm Clarigard, Opticap XL10; 0.22 μm Durapore, Opticap XL10, Merck Millipore, Hellerup, DK) prior to purification.

Large-Scale Expression (CHOK1)—

Suspension-adapted CHOK1 cell lines were cultivated in a chemically defined medium (CDM4CHO, Thermo Scientific HyClone, Fisher Scientific, Slangerup, DK) supplemented with 5 mg/L vitamin K1. During propagation of seed culture for bioreactors the medium was supplemented with 600 μg/ml Geneticin® (Invitrogen by Life Technologies, Naerum, DK). Briefly, the cells were expanded in vented shake-flasks incubated in orbital shakers at 37° C. and 5% CO2. The production phase was performed as a repeated batch culture in bioreactors. pH was controlled around 7 by addition of CO2 and Na2CO3. Dissolved oxygen concentration was kept above 50% of saturation in air by sparging with oxygen. Temperature was maintained at 36.5° C. Withdrawn harvest was clarified through filtration (3 μm Clarigard, Opticap XL10; 0.22 μm Durapore, Opticap XL10, Merck Millipore, Hellerup, DK) prior to purification.

### Example 4

FVII variants were purified by Gla-domain directed antibody affinity chromatography essentially as described elsewhere (Thim et al. 1988). Briefly, the protocol comprised 1 to 3 steps. In step 1, 5 mM CaCl₂ were added to the conditioned medium and the sample loaded onto the affinity column. After extensive wash with 20 mM HEPES, 2 M NaCl, 10 mM CaCl2, 0.005% Tween 80, pH 8.0, bound protein was eluted with 20 mM HEPES, 20 mM NaCl, 20 mM EDTA, 0.005% Tween80, pH 8.0 onto (step 2) an anion exchange column (Source 15Q, GE Healthcare). After wash with 20 mM HEPES, 20 mM NaCl, 0.005% Tween80, pH 8.0, bound protein was eluted with 20 mM HEPES, 135 mM NaCl, 10 mM CaCl2, 0.005% Tween80, pH 8.0 onto (step 3) a CNBr-Sepharose Fast Flow column (GE Healthcare) to which human plasma-derived FXa had been coupled at a density of 1 mg/ml according to manufacturer's instructions. The flow rate was optimized to ensure essentially complete activation of the purified zymogen variants to the activated form. For FVIIa variants with enhanced activity capable of auto-activation in the conditioned medium or on the anion exchange column, step 2 and/or step 3 were omitted to prevent proteolytic degradation. Purified proteins were stored at −80° C. Protein quality was assessed by SDS-PAGE analysis and the concentration of functional molecules measured by active site titration or quantification of the light chain content by rpHPLC as described below.

Measurement of FVIIa Variant Concentration by Active Site Titration—

The concentration of functional molecules in the purified preparations was determined by active site titrationfrom the irreversible loss of amidolytic activity upon titration with sub-stoichiometric levels of d-Phe-Phe-Arg-chloromethyl ketone (FFR-cmk; Bachem) essentially as described elsewhere (Bock, 1992). Briefly, all proteins were diluted in assay buffer (50 mM HEPES (pH 7.4), 100 mM NaCl, 10 mM CaCl2, 1 mg/mL BSA, and 0.1% (w/v) PEG8000). A final concentration of 150 nM FVIIa variant was preincubated with 500 nM soluble tissue factor (sTF) for 10 min followed by the addition of FFR-cmk at final concentrations of 0-300 nM (n=2) in a total reaction volume of 100 μL in a 96-well plate. The reactions were incubated over night at room temperature. In another 96-well plate, 20 μL of each reaction was diluted 10 times in assay buffer containing 1 mM S-2288 (Chromogenix, Milano, Italy). The absorbance increase was measured continuously for 10 min at 405 nM in a Spectramax 190 microplate spectrophotometer equipped with SOFTmax PRO software. Amidolytic activity was reported as the slope of the linear progress curves after blank subtraction. Active site concentrations were determined by extrapolation, as the minimal concentration of FFR-cmk needed to completely abolis the amidolytic activity.

Measurement of FVIIa Variant Concentration from the Light-Chain Content Using Reversed-Phase HPLC—

In alternative approach, the concentration of functional FVIIa molecules in purified preparations were determined by quantification of the FVIIa light chain (LC) content by reversed-phase HPLC (rpHPLC). A calibration curve with wild-type FVIIa was prepared using FVIIa concentrations in the range from 0 to 3 μM, while samples of unknown concentration were prepared in estimated concentrations of 1.5 μM (n=2). All samples were reduced using a 1:1 mixture of 0.5 M tris(2-carboxyethyl)phosphine (TCEP; Calbiochem/Merck KGaA, Darmstadt, Germany) and formic acid added to the samples to a concentration of 20% (v/v) followed by heating of samples at 70° C. for 10 min. The reduced FVIIa variants were loaded onto a C4 column (Vydac, 300 A, particle size 5 μM, 4.6 mm, 250 mm) maintained at 30° C. Mobile phases consisted of 0.09% TFA in water (solvent A) and 0.085% TFA in acetonitrile (solvent B). Following injection of 80 μL sample, the system was run isocratically at 25% solvent B for 4 min followed by a linear gradient from 25-46% B over 10 min. Peaks were detected by fluorescence using excitation and emission wavelengths of 280 and 348 nm, respectively. Light chain quantification was performed by peak integration, and relative amounts of FVIIa variants were calculated on basis of the wild-type FVIIa standard curve.

### Example 5

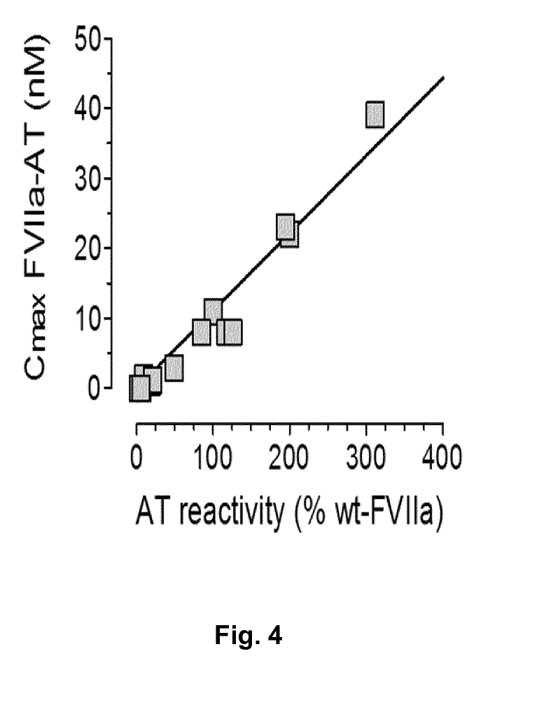

In order to identify FVIIa variants having substantially preserved activity but a reduced reactivity with the plasma inhibitor antithrombin, the generated variant libraries were subjected to the screening assays detailed below, which were established in both manual and automated formats. Briefly, activity was measured as the ability of each variant to proteolytically activate the macromolecular substrate Factor X in the presence of phospholipid vesicles (In vitro proteolysis assay). Each reaction was performed in the presence or absence of the co-factor tissue factor (sTF) to mimic the possible TF dependent and independent mechanisms of action of recombinant FVIIa. The susceptibility of each variant to inhibition by antithrombin was quantified under pseudo-first order conditions in the presence of low molecular weight heparin to mimic the ability of endogenous heparin-like glycosaminoglycans (GAGs) to accelerate the reaction in vivo. As shown in FIG. 4 the measured in vitro antithrombin reactivities were found to correlate with the in vivo accumulation of FVIIa-antithrombin complexes thus validating the predictiveness of the in vitro screening procedure.

Results from the variant screen are given in Table 2 Among the variants, replacement of T293 with Lys (K), Arg (R), Tyr (Y), or Phe (F) reduced the antithrombin reactivity to levels at or below 10% of wild-type FVIIa while the proteolytic activity in the absence of sTF was maintained slightly above wild-type level. For the T293Y variant an activity level >200% of wild-type FVIIa was observed. Similarly, Lys (K), Arg (R), and Asn (N) substitutions at Q176 dramatically reduced the antithrombin reactivity while substantially preserving the proteolytic activity at wild-type levels. Noticeably, an antithrombin reactivity of less than 1% was observed for the Q176R variant.

TABLE 2
Proteolytic activities and antithrombin reactivities of FVIIa variants relative (in %) to wild-type FVIIa.
The proteolytic activity was measured with human plasma-derived FX as substrate in the presence
of phospholipid vesicles and in the presence or absence of soluble tissue factor (sTF) as indicated.
Inhibition by human plasma-derived antithrombin was measured in the presence of low molecular
weight heparin. The expression system used for production of each variant is indicated.

| | | Proteolytic | Proteolytic | Anti- | |
|---|---|---|---|---|---|
| | | activity | activity | thrombin | |
| | Sequence | +PS:PC | +sTF/PS: | inhibition | Expression |
| Protein name | modifications | (%) | PC (%) | (%) | system |
| FVIIa A175K | 175A>K | 123 | 113.5 | 125.6 | BHK |
| FVIIa V172N | 172V>N | 76.5 | 94.4 | 28.4 | BHK |
| FVIIa N173D | 173N>D | 35.1 | 51.5 | 80.9 | BHK |
| FVIIa G174E | 174G>E | 49 | 62.9 | 118.4 | BHK |
| FVIIa G174S | 174G>S | 62.2 | 68.8 | 131.2 | BHK |
| FVIIa A175L | 175A>L | 67.9 | 64.5 | 169.6 | BHK |
| FVIIa Q176K | 176Q>K | 80.4 | 116.9 | 7.2 | BHK |
| FVIIa Q286N | 286Q>N | 29.7 | 83.3 | 5.8 | BHK |
| FVIIa Q176L | 176Q>L | 66.2 | 63.4 | 144.1 | BHK |
| FVIIa K197N | 197K>N | 45.8 | 66.4 | 99 | BHK |
| FVIIa K199G | 199K>G | 60 | 76.3 | 81.1 | BHK |
| FVIIa K199S | 199K>S | 67.4 | 88.4 | 91.1 | BHK |
| FVIIa N200Y | 200N>Y | 93.2 | 101.9 | 134.1 | BHK |
| FVIIa R202A | 202R>A | 27.4 | 51.6 | 86.3 | BHK |
| FVIIa R202V | 202R>V | 25.4 | 54.1 | 98.2 | BHK |
| FVIIa N203K | 203N>K | 122.4 | 103.2 | 179.9 | BHK |
| FVIIa 1205T | 205I>T | 41.6 | 62.8 | 67.5 | BHK |
| FVIIa V235T | 235V>T | 72.1 | 86.1 | 112.4 | BHK |
| FVIIa P236A | 236P>A | 52.9 | 73.1 | 91.2 | BHK |
| FVIIa T238Q | 238T>Q | 24.5 | 32.1 | 40.4 | BHK |
| FVIIa t239A | 239T>A | 100.8 | 102.7 | 160.7 | BHK |
| FVIIa T239I | 239T>I | 22.8 | 53.5 | 10.9 | BHK |
| FVIIa Q286V | 286Q>V | 10.9 | 31.6 | 18.7 | BHK |
| FVIIa Q286T | 286Q>T | 3.6 | 13.2 | 17.7 | BHK |
| FVIIa Q286L | 286Q>L | 13.8 | 31.3 | 27.1 | BHK |
| FVIIa A292V | 292A>V | 56.4 | 71.3 | 165.5 | BHK |
| FVIIa T293K | 293T>K | 112 | 140.6 | 2.7 | BHK |
| FVIIa T293L | 293T>L | 175.5 | 117.3 | 108.1 | BHK |
| FVIIa L295R | 295L>R | 4 | 13.8 | 10.4 | BHK |
| FVIIa D319A | 319D>A | 176.9 | 86.1 | 163.6 | BHK |
| FVIIa D319G | 319D>G | 77.3 | 109.8 | 74.8 | BHK |
| FVIIa A175T | 175A>T | 12.9 | 29.3 | 57 | BHK |
| FVIIa A292R | 292A>R | 45.8 | 62.4 | 194.3 | BHK |
| FVIIa Q176A | 176Q>A | 34.6 | 67.2 | 9.6 | BHK |
| FVIIa Q176N | 176Q>N | 104.5 | 132.8 | 15.9 | BHK |
| FVIIa Q176S | 176Q>S | 43.9 | 87.3 | 19.2 | BHK |
| FVIIa I198A | 198I>A | 71.2 | 66.4 | 44.9 | BHK |
| FVIIa K199A | 199K>A | 40.6 | 57.3 | 130.6 | BHK |
| FVIIa T293A | 293T>A | 25 | 54.4 | 13.5 | BHK |
| FVIIa T293S | 293T>S | 68.3 | 111.6 | 25.4 | BHK |
| FVIIa Q286R | 286Q>R | 150.6 | 235.5 | 31.2 | BHK |
| FVIIa A292E | 292A>E | 5.6 | 8.6 | 29.7 | BHK |
| FVIIa M298Q | 298M>Q | 625.5 | 252.2 | 200.1 | BHK |
| FVIIa Q366E | 366Q>E | 82.9 | 55.8 | 60.8 | HEK293 |
| FVIIa Q366D | 366Q>D | 20.5 | 10.1 | 10.5 | HEK293 |
| FVIIa T370A | 370T>A | 67.3 | 121.9 | 158.9 | HEK293 |
| FVIIa V371E | 371V>E | 20 | 68.4 | 4 | HEK293 |
| FVIIa V371T | 371V>T | 10.4 | 79 | 7.6 | HEK293 |
| FVIIa V371I | 371V>I | 87.5 | 71.4 | 117.3 | HEK293 |
| FVIIa A175T | 175A>T | 21.6 | 21 | 35.2 | HEK293 |
| FVIIa I205T | 205I>T | 41.2 | 77.8 | 61.6 | HKB11 |
| FVIIa P236N | 236P>N | −0.1 | 4.5 | 70.3 | HKB11 |
| FVIIa G237N | 237G>N | 4.8 | 7 | 29.2 | HKB11 |
| FVIIa G291N | 291G>N | −1.1 | 0.1 | 0 | HKB11 |
| FVIIa Q286A | 286Q>A | 44.3 | 63.2 | 88.2 | BHK |
| FVIIa Q286Y | 286Q>Y | 4.6 | 24.3 | 7.4 | BHK |
| FVIIa I198Q | 198I>Q | 32.8 | 43.8 | 35.5 | BHK |
| FVIIa I198E | 198I>E | 76.4 | 69.6 | 82.6 | BHK |
| FVIIa Q286I | 286Q>I | −0.2 | 3.5 | 4.4 | CHO |
| FVIIa Q286K | 286Q>K | 22.6 | 63.8 | 54.8 | BHK |
| FVIIa Q286W | 286Q>W | 13.9 | 42.1 | 9.6 | BHK |
| FVIIa Q286R | 286Q>R | 97.7 | 111.9 | 27.9 | BHK |
| FVIIa Q176H | 176Q>H | 18.7 | 46 | 7.9 | BHK |
| FVIIa K199H | 199K>H | 34.3 | 45.6 | 16.3 | BHK |
| FVIIa Q286F | 286Q>F | 17.7 | 39.2 | 6.4 | HKB11 |
| FVIIa Q286M | 286Q>M | 163.8 | 196.7 | 30.6 | HKB11 |
| FVIIa Q286D | 286Q>D | 0.6 | 5.6 | 0 | HKB11 |
| FVIIa Q286E | 286Q>E | 9.6 | 3.8 | 12.6 | HKB11 |
| FVIIa Q176R | 176Q>R | 68.1 | 111.3 | 0.8 | HKB11 |
| FVIIa A175Q | 175A>Q | 73.6 | 65.2 | 117.4 | HKB11 |
| FVIIa Q176I | 176Q>I | 24.1 | 41.6 | 41.6 | HKB11 |
| FVIIa A175F | 175A>F | 111.2 | 73.1 | 89.8 | HKB11 |
| FVIIa A175H | 175A>H | 143.3 | 113 | 100.7 | HKB11 |
| FVIIa A175V | 175A>V | 88.4 | 94.8 | 87.3 | HKB11 |
| FVIIa Q176E | 176Q>E | 10.5 | 32.3 | 9 | CHO |
| FVIIa Q176F | 176Q>F | 24.3 | 63.6 | 2.5 | HKB11 |
| FVIIa Q176T | 176Q>T | 43.7 | 65.3 | 21.2 | CHO |
| FVIIa Q176V | 176Q>V | 19.7 | 32.1 | 0 | HKB11 |
| FVIIa Q176Y | 176Q>Y | 11.9 | 22.9 | 0 | CHO |
| FVIIa T293M | 293T>M | 118 | 75.6 | 36.6 | HKB11 |
| FVIIa T293F | 293T>F | 132.4 | 76 | 7.7 | CHO |
| FVIIa Q176M | 176Q>M | 103.4 | 87.9 | 49 | HKB11 |
| FVIIa I198S | 198I>S | 76 | 88.7 | 47.4 | HKB11 |
| FVIIa I198T | 198I>T | 7.4 | 83.4 | 19.9 | HKB11 |
| FVIIa A175D | 175A>D | 84.9 | 86.1 | 65.3 | HKB11 |
| FVIIa Q176W | 176Q>W | 3.3 | 40.5 | 1.1 | CHO |
| FVIIa Q286H | 286Q>H | 3.3 | 6.8 | 5 | CHO |
| FVIIa Q286S | 286Q>S | 20.7 | 38.2 | 32.8 | CHO |
| FVIIa T293D | 293T>D | 3.4 | 13 | 0.5 | CHO |
| FVIIa T293I | 293T>I | 19.6 | 43.1 | 12.1 | CHO |
| FVIIa T293N | 293T>N | 49.1 | 54 | 20.1 | CHO |
| FVIIa T293H | 293T>H | 51 | 65.4 | 6.5 | HKB11 |
| FVIIa A175Y | 175A>Y | 127.9 | 106.6 | 84.5 | CHO |
| FVIIa A175N | 175A>N | 82.2 | 89.5 | 139.3 | CHO |
| FVIIa V172D | 172V>D | 52.8 | 65 | 24.5 | CHO |
| FVIIa A175R | 175A>R | 110.6 | 83.1 | 125.1 | CHO |
| FVIIa A175W | 175A>W | 124.7 | 111.7 | 76.9 | CHO |
| FVIIa I198V | 198I>V | 45.2 | 54.4 | 80.7 | CHO |
| FVIIa T293Y | 293T>Y | 237.2 | 92.1 | 10.3 | CHO |
| FVIIa T293W | 293T>W | 8.4 | 5.5 | 6.4 | CHO |
| FVIIa A175S | 175A>S | 20.6 | 28.6 | 46.7 | CHO |
| FVIIa V172K | 172V>K | 95.1 | 83 | 87.9 | CHO |
| FVIIa V172E | 172V>E | 32.3 | 43.1 | 27.5 | CHO |
| FVIIa V172M | 172V>M | 61.9 | 68.9 | 60.8 | CHO |
| FVIIa V172Q | 172V>Q | 16.1 | 24.7 | 13.2 | CHO |
| FVIIa V172T | 172V>T | 65.5 | 55.1 | 72.8 | CHO |
| FVIIa V172W | 172V>W | 39.1 | 49.3 | 72.3 | CHO |
| FVIIa T293R | 293T>R | 133.2 | 128.9 | 3.8 | CHO |
| FVIIa T293V | 293T>V | 25.3 | 57.7 | 17.6 | CHO |
| FVIIa V172L | 172V>L | 65.7 | 64.4 | 72.7 | CHO |
| FVIIa V172G | 172V>G | 28.8 | 57 | 26.3 | CHO |
| FVIIa I198L | 198I>L | 50.5 | 66.3 | 80.1 | CHO |
| FVIIa V172Y | 172V>Y | 72.4 | 67.5 | 86.4 | CHO |
| FVIIa V172I | 172V>I | 63 | 64.5 | 83.4 | CHO |
| FVIIa V172A | 172V>A | 54.6 | 61.9 | 93.2 | CHO |
| FVIIa V172S | 172V>S | 57.9 | 56.9 | 56.6 | CHO |
| FVIIa A175M | 175A>M | 84.3 | 96 | 111.3 | CHO |
| FVIIa V172R | 172V>R | 122.1 | 111.1 | 47.8 | CHO |
| FVIIa M298Q | 298M>Q | 625.5 | 252.2 | 200.1 | BHK |
| FVIIa K337A | 337K>A | 214.5 | 122 | 107.3 | CHO |
| FVIIa Q176A M298Q | 176Q>A, 298M>Q | | | | CHO |
| FVIIa Q176L M298Q | 176Q>L, 298M>Q | | | | CHO |
| FVIIa V158D Q176L | 158V>D, 176Q>L, | | | | CHO |
| E296V M298Q | 296E>V, 298M>Q | | | | |
| FVIIa Q286A M298Q | 286Q>A, 298M>Q | | | | CHO |
| FVIIa V158D Q286A | 158V>D, 286Q>A, | | | | CHO |
| E296V M298Q | 296E>V, 298M>Q | | | | |
| FVIIa V158D Q286R | 158V>D, 286Q>R, | 201.9 | 131.5 | 50.1 | CHO |
| E296V M298Q | 296E>V, 298M>Q | | | | |
| FVIIa T293A M298Q | 293T>A, 298M>Q | 8964.3 | 340.2 | 205.6 | CHO |
| FVIIa T293L M298Q | 293T>L, 298M>Q | 1542.5 | 218.5 | 189.5 | CHO |
| FVIIa V158D T293L | 158V>D, 293T>L, | | | | CHO |
| E296V M298Q | 296E>V, 298M>Q | | | | |
| FVIIa V158D Q286N | 158V>D, 286Q>N, | 2771.2 | 594.5 | 84.7 | CHO |
| E296V M298Q | 296E>V, 298M>Q | | | | |
| FVIIa Q286N M298Q | 286Q>N, 298M>Q | 137.6 | 128.2 | 20.9 | CHO |
| FVIIa V158D Q176K | 158V>D, 176Q>K, | 7562.8 | 447.3 | 49.1 | CHO |
| E296V M298Q | 296E>V, 298M>Q | | | | |
| FVII Q176K M298Q | 176Q>K, 298M>Q | 584.9 | 214.4 | 9.6 | CHO |
| FVIIa V158D T293K | 158V>D, 293T>K, | 5406.9 | 232.8 | 19.5 | CHO |
| E296V M298Q | 296E>V, 298M>Q | | | | |
| FVIIa T293K M298Q | 293T>K, 298M>Q | 1025.5 | 512.9 | 7.5 | CHO |
| FVIIa V158D E296V | 158V>D, 296E>V, | 8916.7 | 517 | 311.7 | CHO |
| M298Q | 298M>Q | | | | |
| FVIIa L305V S314E | 305L>V, 314S>E, | 426.3 | 92.2 | 315.8 | CHO |
| K337A F374Y | 337K>A, 374F>Y | | | | |
| FVIIa Q176K L305V | 176Q>K, 305L>V, | 1279.8 | 127.6 | 83.5 | CHO |
| S314E K337A F374Y | 314S>E, 337K>A, | | | | |
| | 374F>Y | | | | |
| FVIIa Q286N L305V | 286Q>N, 305L>V, | 883.2 | 96.9 | 141.1 | CHO |
| S314E K337A F374Y | 314S>E, 337K>A, | | | | |
| | 374F>Y | | | | |
| FVIIa T293K L305V | 293T>K, 305L>V, | 1519.7 | 198.3 | 69.4 | CHO |
| S314E K337A F374Y | 314S>E, 337K>A, | | | | |
| | 374F>Y | | | | |

Measurement of Proteolytic Activity Using Factor X as Substrate (Manual In Vitro Proteolysis Assay)—

The proteolytic activity of the FVIIa variants was estimated using plasma-derived factor X (FX) as substrate. All proteins were diluted in assay buffer (50 mM HEPES (pH 7.4), 100 mM NaCl, 10 mM CaCl₂, 1 mg/mL BSA, and 0.1% (w/v) PEG8000). The kinetic parameters for FX activation were determined by incubating 10 nM of each FVIIa conjugate with 40 nM FX in the presence of 25 μM 75:25 phosphatidyl choline:phosphatidyl serine phospholipid vesicles (PS:PC; Haematologic Technologies, Vermont, USA) for 30 min at room temperature in a total reaction volume of 100 μl in a 96-well plate (n=2). FX activation in the presence of sTF was determined by incubating 5 μM of each FVIIa conjugate with 30 nM FX in the presence of 25 μM PC:PS phospholipids for 20 minutes at room temperature in a total reaction volume of 100 μl (n=2). After incubation, reactions were quenched by adding 50 μl stop buffer [50 mM HEPES (pH 7.4), 100 mM NaCl, 80 mM EDTA] followed by the addition of 50 μl 2 mM S-2765 chromogenic substrate (Chromogenix, Milano, Italy). Finally, the absorbance increase was measured continuously at 405 nM in a Spectramax 190 microplate reader. Apparent catalytic rate values (k_{cat}/Kₘ) were estimated by fitting the data to a simplified form of the Michaelis Menten equation (v=kcat*[S]*[E]/Km) using linear regression since the FX substrate concentration ([S]) was below Km for the activation reaction. The amount of FXa generated was estimated from a standard curve prepared with human plasma-derived FXa under identical conditions. Estimated k_{cat}/kₘ values were reported relative to that of wild-type FVIIa. Results are given in Table 2 and Table 3.

TABLE 3
In vitro functional (upper part) and pharmacokinetic (lower part) properties of
FVIIa variants and PEGylated conjugates

| | | | FVIIa clot | Proteolytic | TEG R-time EC50 | TGA potency | AT inhibition |
|---|---|---|---|---|---|---|---|
| Compound | | | potency (%) | activity (%) | (nM) | (%) | (%) |
| FVIIa | | | 100 ± 7 | 100 | 0.43 ± 0 | 100 | 100 |
| | | +40k PEG | 15 ± 1 | 25 ± 4 | 1.80 ± 1 | 40 ± 6 | 63 ± 10 |
| | Q286N | | 65 ± 7 | 30 ± 1 | 0.54 | 27 ± 4 | 6 ± 1 |
| | | +40k PEG | 7.1 ± 1 | 32 ± 1 | | | 2 ± 1 |
| | Q176K | | 115 ± 22 | 80 ± 4 | 0.84 ± 0 | 135 ± 49 | 7 ± 3 |
| | T293K | | 140 ± 15 | 112 ± 6 | 0.46 ± 0 | 135 ± 49 | 3 ± 3 |
| M298Q | | | 182 ± 23 | 626 ± 39 | | | 200 ± 74 |
| | Q286N | | 135 ± 5 | 138 ± 7 | | | 21 ± 4 |
| | | +40k PEG | 26 ± 2 | 47 ± 1 | | | 12 ± 3 |
| | Q176K | | 274 ± 7 | 585 ± 6 | | | 10 ± 5 |
| | | +40k PEG | 43 ± 4 | 191 ± 9 | | | 6 ± 8 |
| | T293K | | 242 ± 66 | 1026 ± 13 | | | 8 ± 2 |
| | | +40k PEG | 45 ± 8 | 222 ± 9 | 0.52 ± 0 | | 5.6 ± 2 |
| DVQ | | | 480 ± 43 | 8917 ± 239 | 0.07 ± 0 | 4000 ± 600 | 312 ± 104 |
| | | +40k PEG | 68 ± 11 | 917 ± 47 | | | 210 ± 62 |
| | Q286N | | 310 ± 51 | 2771 ± 35 | 0.06 ± 0 | 3900 ± 570 | 85 ± 20 |
| | | +40k PEG | 110 ± 14 | 1042 ± 37 | | | 65 ± 14 |
| | Q176K | | 403 ± 25 | 7563 ± 48 | 0.17 ± 0 | 3900 ± 570 | 49 ± 21 |
| | | +40k PEG | 89 ± 17 | 1488 ± 24 | | | 31 ± 19 |
| | T293K | | 185 ± 8 | 5407 ± 151 | | | 20 ± 11 |

| | | | T½ in rat | | Rat AT complex | T½ in dog | MRT in dog | | Dog AT complex |
|---|---|---|---|---|---|---|---|---|---|
| Compound | | | (h) | MRT in rat (h) | Cmax/dose (Kg/L) | (h) | (h) | | Cmax/dose (Kg/L) |
| FVIIa | | | 0.8 ± 0.01 | 1.1 ± 0.03 | 0.6 ± 0.08 | 1.7 ± 0.0 | 2.2 | 0.1 | 2.7 ± 0.20 |
| | | +40k PEG | 7.4 ± 0.20 | 8.3 ± 0.30 | 0.7 ± 0.05 | 8 ± 0.2 | 11 | 0.4 | 2.9 ± 0.40 |
| | Q286N | | 1.0 ± 0.02 | 1.2 ± 0.04 | <LLOQ | 2.3 ± 0.1 | 2.7 | 0.2 | 0.9 ± 0.10 |
| | | +40k PEG | 16 ± 0.20 | 22 ± 0.80 | <LLOQ | 20 ± 2.1 | 28 | 3.5 | 0.4 ± 0.07 |
| | Q176K | | 1.0 ± 0.03 | 1.0 ± 0.06 | <LLOQ | | | | |
| | T293K | | 1.1 ± 0.00 | 1.2 ± 0.02 | <LLOQ | | | | |
| M298Q | | | 0.9 ± 0.02 | 1.0 ± 0.01 | 1.12 ± 0.17 | | | | |
| | Q286N | | 0.9 ± 0.02 | 1.1 ± 0.10 | 0.06 ± 0.01 | | | | |
| | | +40k PEG | 12 ± 1.10 | 15 ± 1.00 | 0.08 ± 0.02 | | | | |
| | Q176K | | 0.9 ± 0.02 | 1.1 ± 0.03 | <LLOQ | 1.6 ± 0.2 | 2.1 | 0.31 | 0.6 ± 0.06 |
| | | +40k PEG | 7.8 ± 0.20 | 8.2 ± 0.10 | <LLOQ | 17 ± 0.6 | 18 | 0.89 | [symbol] LLOQ |
| | T293K | | 1.0 ± 0.02 | 1.1 ± 0.03 | <LLOQ | | | | |
| | | +40k PEG | 13 ± 0.30 | 15 ± 0.60 | 0.07 ± 0.03 | | | | |
| DVQ | | | 1.2 ± 0.10 | 0.7 ± 0.10 | 1.9 ± 0.30 | | | | |
| | | +40k PEG | 7.2 ± 1.30 | 2.1 ± 0.30 | 2.6 ± 0.30 | 8.6 ± 1.2 | 2.6 | 0.4 | 4.6 ± 0.50 |
| | Q286N | | 0.8 ± 0.02 | 0.8 ± 0.04 | 0.40 ± 0.06 | | | | |
| | | +40k PEG | 5.6 ± 0.10 | 5.5 ± 0.20 | 0.30 ± 0.02 | 6.3 ± 0.3 | 6.4 | 0.4 | 1.7 ± 0.20 |
| | Q176K | | 0.6 ± 0.01 | 0.7 ± 0.04 | 0.10 ± 0.02 | 1.2 ± 0.2 | 1.0 | 0.08 | 1.1 ± 0.10 |
| | | +40k PEG | 3.9 ± 0.04 | 3.1 ± 0.06 | 0.20 ± 0.10 | 2.0 ± 0.1 | 2.4 | 0.08 | 1.1 ± 0.01 |
| | T293K | | 0.7 ± 0.04 | 0.8 ± 0.06 | 0.05 ± 0.01 | | | | |

| | FVIIa clot | Proteolytic | TEG R-time | TGA potency | AT inhibition |
|---|---|---|---|---|---|
| Compound | potency (%) | activity (%) | EC50 (nM) | (%) | (%) |
| 40-HEP-[C]-FVIIa T293K 407C | 27 ± 2 | 72 ± 5 | | | 2 ± 1 |

| | | | Rat AT complex Cmax/dos |
|---|---|---|---|
| Compound | T½ in rat (h) | MRT in rat (h) | (Kg/L) |
| 40-HEP-[C]-FVIIa T293K 407C | 17.3 ± 0.5 | 21.9 ± 1 | <LLOQ |

DVQ: FVIIa V158D/E296V/M298Q
AT: antithrombin
+40k PEG: GlycoPEGylated with a 40-kDa PEG.
FVIIa clot potency: sTF dependent specific clot activity in FVII deficient plasma in percent of wild-type FVIIa specific activity.
Proteolytic activity: FXa activity in the presence of PS:PC vesicles in percent of wild-type FVIIa.
TEG R-time: kaolin induced thromboelastography clot time of haemophilia like human whole blood.
TGA potency: Rate of thrombin generation in platelet rich haemophilia like plasma in percent of wild-type rFVIIa
AT inhibition: inhibition by AT in the presence of low molecular weight thrombin in percent of wild type rFVIIa.
T½: Terminal half-life of the active molecule following IV administration
MRT: Mean residence time of the active molecule following IV administration.
AT complex Cmax/dose: Maximum measured level of compound-antithrombin complex divided by the dose. [symbol]
indicates data missing or illegible when filed

Measurement of Proteolytic Activity Using Factor X as Substrate (Automated In Vitro Proteolysis Assay)—

The proteolytic activity of the FVIIa variants was estimated using plasma-derived factor X (FX) as substrate. All proteins were diluted in 50 mM HEPES (pH 7.4), 100 mM NaCl, 10 mM CaCl₂, 1 mg/mL BSA, and 0.1% (w/v) PEG8000. Relative proteolytic activities were determined by incubating 10 nM of each FVIIa conjugate with 40 nM FX in the presence of 25 μM 75:25 phosphatidyl choline:phosphatidyl serine (PC:PS) phospholipids (Haematologic technologies, Vermont, USA) for 30 min at room temperature in a total reaction volume of 100 μL in a 96-well plate (n=2). FX activation in the presence of sTF was determined by incubating 5 μM of each FVIIa conjugate with 30 nM FX in the presence of 25 μM PC:PS phospholipids for 20 min at room temperature in a total reaction volume of 100 μL (n=2). After incubation, reactions were quenched by adding 100 μL 1 mM S-2765 in stop buffer [50 mM HEPES (pH 7.4), 100 mM NaCl, 80 mM EDTA]. Immediately after quenching, the absorbance increase was measured continuously at 405 nM in an Envision microplate reader (PerkinElmer, Waltham, Mass.). All additions, incubations and plate movements were performed by a Hamilton Microlab Star robot robot (Hamilton, Bonaduz, Switzerland) on line coupled to an Envision microplate reader. Proteolytic activities are calculated relative to wild-type FVIIa. Results are given in Table 2 and Table 3

Measurement of FVIIa Inhibition by Antithrombin (Manual Assay)—

A discontinuous method was used to measure the in vitro rate of inhibition by human plasma-derived antithrombin (AT) under pseudo-first order conditions in the presence of low molecular weight (LMW) heparin (Calbiochem/Merck KGaA, Darmstadt, Germany). The assay was performed in a 96-well plate using an assay buffer containing 50 mM HEPES (pH 7.4), 100 mM NaCl, 10 mM CaCl₂, 1 mg/mL BSA, and 0.1% (w/v) PEG8000 in a total reaction volume of 200 μl. To a mixture of 200 nM FVIIa and 12 μM LMW heparin was added 5 μM antithrombin in a final reaction volume of 100 μl. At different times, the reaction was quenched by transferring 20 μl of the reaction mixture to another microtiter plate containing 180 μL of sTF (200 nM), polybrene (0.5 mg/ml; Hexadimethrine bromide, Sigma-Aldrich) to quench the reaction, and S-2288 (1 mM). Immediately after transfer at the different times, substrate cleavage was monitored at 405 nm for 10 min in a Spectramax 190 microplate reader. Pseudo-first order rate constants (kobs) were obtained by non-linear least-squares fitting of data to an exponential decay function, and the second-order rate constant (k) was obtained from the following relationship k=kobs/[AT]. Rates of inhibition were reported relative to that of wild-type FVIIa. Results are given in Table 2 and Table 3.

Measurement of FVIIa Inhibition by Antithrombin (Automated Assay)—

A discontinuous method was used to measure the in vitro rate of inhibition by human plasma-derived antithrombin (AT) under pseudo-first order conditions in the presence of low molecular weight (LMW) heparin (Calbiochem/Merck KGaA, Darmstadt, Germany). The assay was performed in a 96-well plate using a buffer containing 50 mM HEPES (pH 7.4), 100 mM NaCl, 10 mM CaCl2, 1 mg/mL BSA, and 0.1% (w/v) PEG8000 in a total reaction volume of 200 μL. To a mixture of 200 nM FVIIa and 12 μM LMW heparin was added 5 μM antithrombin in a final reaction volume of 100 μL. At different times, the reaction was quenched by transferring 20 μL of the reaction mixture to another microtiter plate containing 180 μL of sTF (200 nM), polybrene (0.5 mg/mL; Hexadimethrine bromide, Sigma-Aldrich) and S-2288 (1 mM). Immediately after transfer at the different times, substrate cleavage was monitored at 405 nm for 10 min in an Envision microplate reader. Pseudo-first order rate constants (kobs) were obtained by non-linear least-squares fitting of data to an exponential decay function, and the second-order rate constant (k) was obtained from the following relationship k=kobs/[AT]. All additions, incubations and plate movements were performed by a Hamilton Microlab Star robot (Hamilton, Bonaduz, Switzerland) on line coupled to an Envision microplate reader (PerkinElmer, Waltham, Mass.). Rates of inhibition were reported relative to that of wild-type FVIIa. Rates of inhibition were reported relative to that of wild-type FVIIa. Results are given in Table 2 and Table 3.

### Example 6

A selection of the identified antithrombin resistant FVIIa variants was further evaluated in combination with the activity enhancing substitutions M298Q, V158D/E296V/M298Q, or L305V/S314E/K337A/F374Y.

Characterization of purified protein preparations using the proteolytic assay described in Example 5 demonstrated that the variants retained super activity. This was confirmed by potency estimation using the STACLOT®VIIa-rTF plasma-based assay described in Example 7 and for some variants also by thrombin generation and thromboelastography in FVIII-deficient plasma as described in Example 8 (see Table 2. In addition, the T293K and Q176K mutations effectively reduced the antithrombin reactivity of M298Q to below 10% of wild-type FVIIa, while a less pronounced reduction was observed in combination with the more active (and antithrombin reactive) variants V158D/E296V/M298Q or L305V/S314E/K337A/F374Y (see Table 4). In the V158D/E296V/M298Q background, the T293K mutation reduced the antithrombin reactivity to about 20% of wild-type levels. Conversely, neither T293A nor T293L were capable of maintaining the antithrombin reactivity below 100% in the M298Q background. These data show that the Q176K and T293K mutations are superior in terms of maintaining activity while substantially reducing the antithrombin reactivity.

TABLE 4
Proteolytic activities and antithrombin reactivities (in % of wild-type) of FVIIa variants in combination with
the activity enhancing substitutions M298Q, V158D/E296V/M298Q, or L305V/S314E/K337A/F374Y.
The proteolytic activity was measured with human plasma-derived FX as substrate in the presence of
phospholipid vesicles and in the presence or absence of soluble tissue factor (sTF) as indicated. Inhibition
by human plasma-derived antithrombin was measured in the presence of low molecular weight heparin.
The expression system used for production of each variant is indicated.

| | | | Proteolytic | Anti- | |
|---|---|---|---|---|---|
| | | Proteolytic | activity | thrombin | |
| | | activity | +sTF/PS:PC | inhibition | Expression |
| Protein name | Sequence modifications | +PS:PC (%) | (%) | (%) | system |
| FVIIa Q176A M298Q | 176Q>A, 298M>Q | | | | CHO |
| FVIIa Q176L M298Q | 176Q>L, 298M>Q | | | | CHO |
| FVIIa V158D Q176L | 158V>D, 176Q>L, | | | | CHO |
| E296V M298Q | 296E>V, 298M>Q | | | | |
| FVIIa Q286A M298Q | 286Q>A, 298M>Q | | | | CHO |
| FVIIa V158D Q286A | 158V>D, 286Q>A, | | | | CHO |
| E296V M298Q | 296E>V, 298M>Q | | | | |
| FVIIa T293A M298Q | 293T>A, 298M>Q | 8964.3 | 340.2 | 205.6 | CHO |
| FVIIa T293L M298Q | 293T>L, 298M>Q | 1542.5 | 218.5 | 189.5 | CHO |
| FVIIa V158D T293L | 158V>D, 293T>L, | | | | CHO |
| E296V M298Q | 296E>V, 298M>Q | | | | |
| FVIIa V158D Q286N | 158V>D, 286Q>N, | 2771.2 | 594.5 | 84.7 | CHO |
| E296V M298Q | 296E>V, 298M>Q | | | | |
| FVIIa Q286N M298Q | 286Q>N, 298M>Q | 137.6 | 128.2 | 20.9 | CHO |
| FVIIa V158D Q176K | 158V>D, 176Q>K, | 7562.8 | 447.3 | 49.1 | CHO |
| E296V M298Q | 296E>V, 298M>Q | | | | |
| FVII Q176K M298Q | 176Q>K, 298M>Q | 584.9 | 214.4 | 9.6 | CHO |
| FVIIa V158D T293K | 158V>D, 293T>K, | 5406.9 | 232.8 | 19.5 | CHO |
| E296V M298Q | 296E>V, 298M>Q | | | | |
| FVIIa T293K M298Q | 293T>K, 298M>Q | 1025.5 | 512.9 | 7.5 | CHO |
| FVIIa V158D E296V | 158V>D, 296E>V, | 8916.7 | 517 | 311.7 | CHO |
| M298Q | 298M>Q | | | | |
| FVIIa L305V S314E | 305L>V, 314S>E, | 426.3 | 92.2 | 315.8 | CHO |
| K337A F374Y | 337K>A, 374F>Y | | | | |
| FVIIa Q176K L305V | 176Q>K, 305L>V, | 1279.8 | 127.6 | 83.5 | CHO |
| S314E K337A F374Y | 314S>E, 337K>A, | | | | |
| | 374F>Y | | | | |
| FVIIa Q286N L305V | 286Q>N, 305L>V, | 883.2 | 96.9 | 141.1 | CHO |
| S314E K337A F374Y | 314S>E, 337K>A, | | | | |
| | 374F>Y | | | | |
| FVIIa T293K L305V | 293T>K, 305L>V, | 1519.7 | 198.3 | 69.4 | CHO |
| S314E K337A F374Y | 314S>E, 337K>A, | | | | |
| | 374F>Y | | | | |

### Example 7

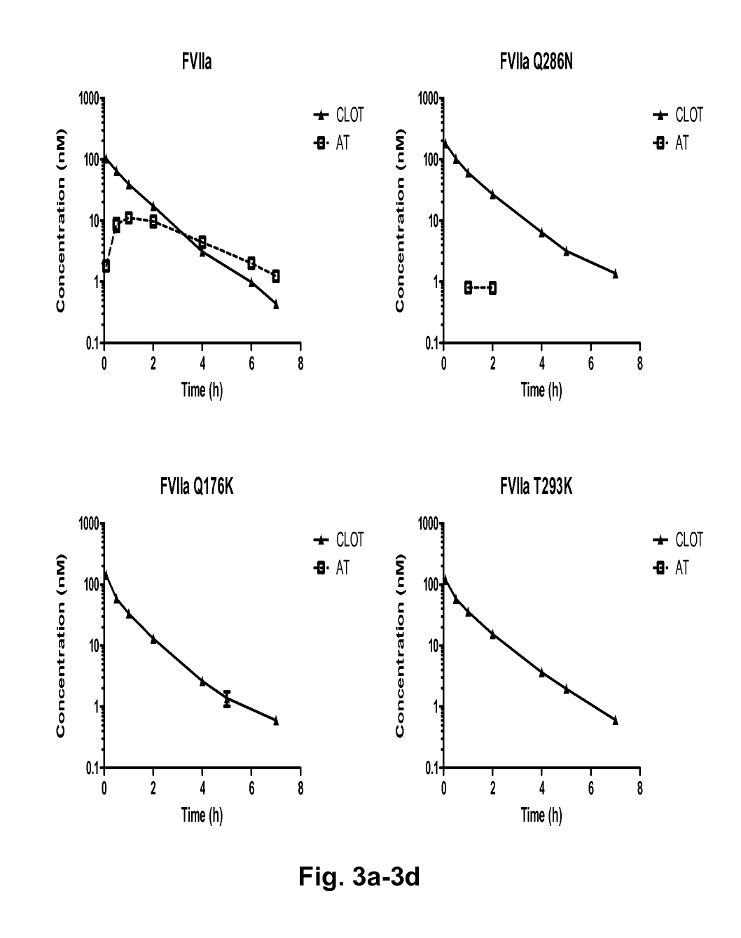

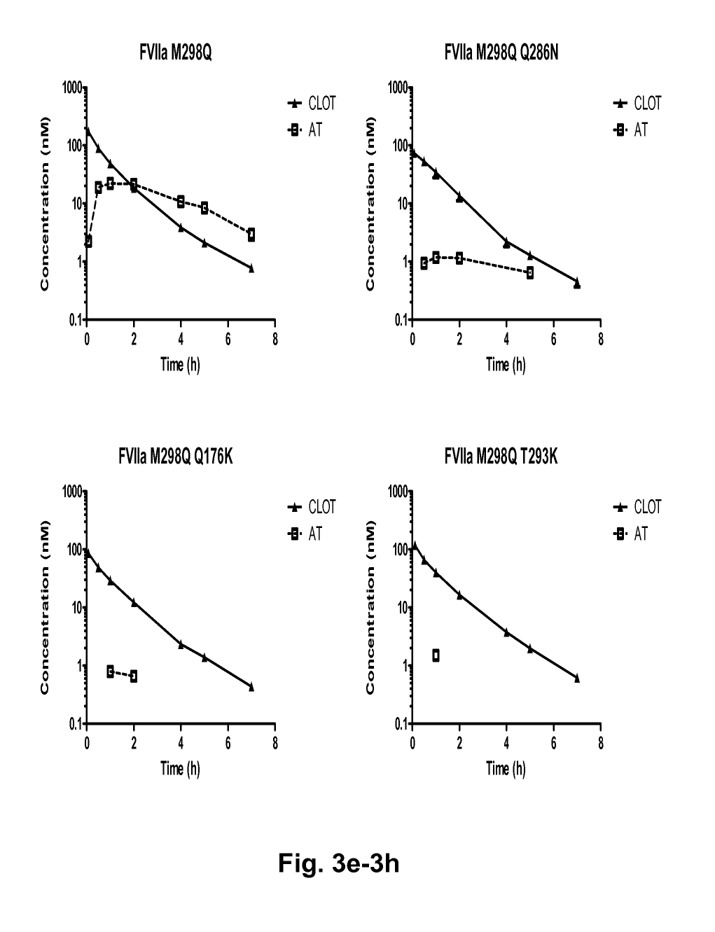

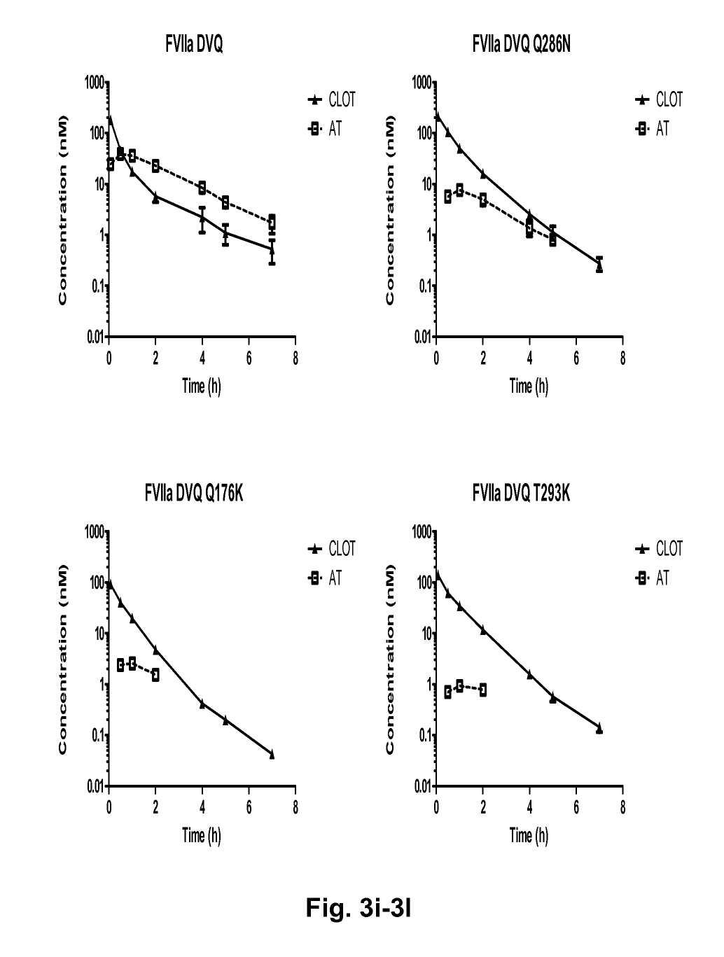

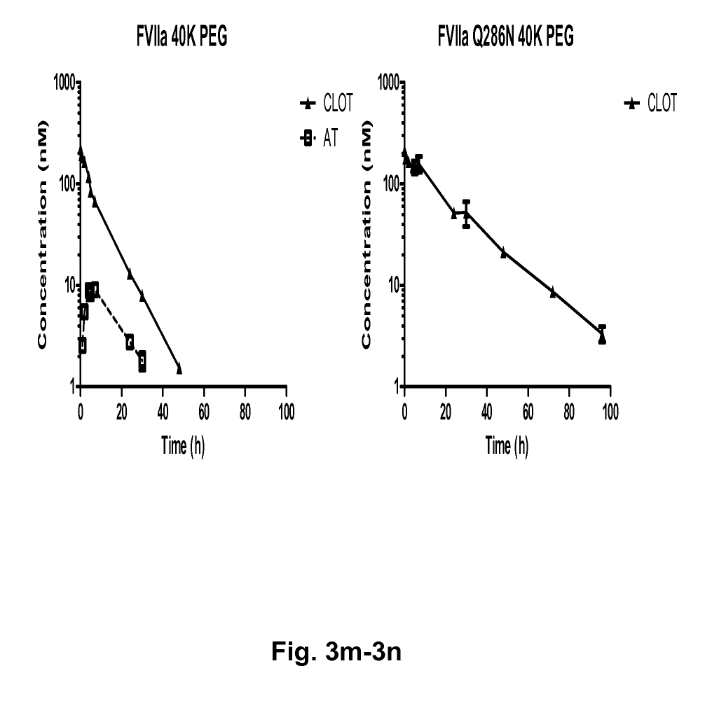

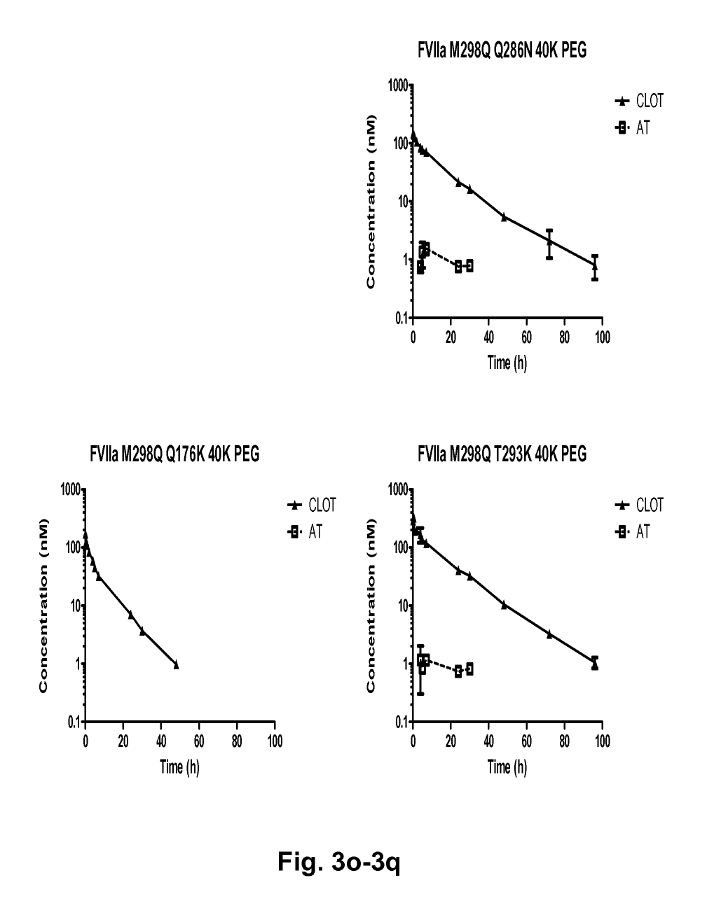

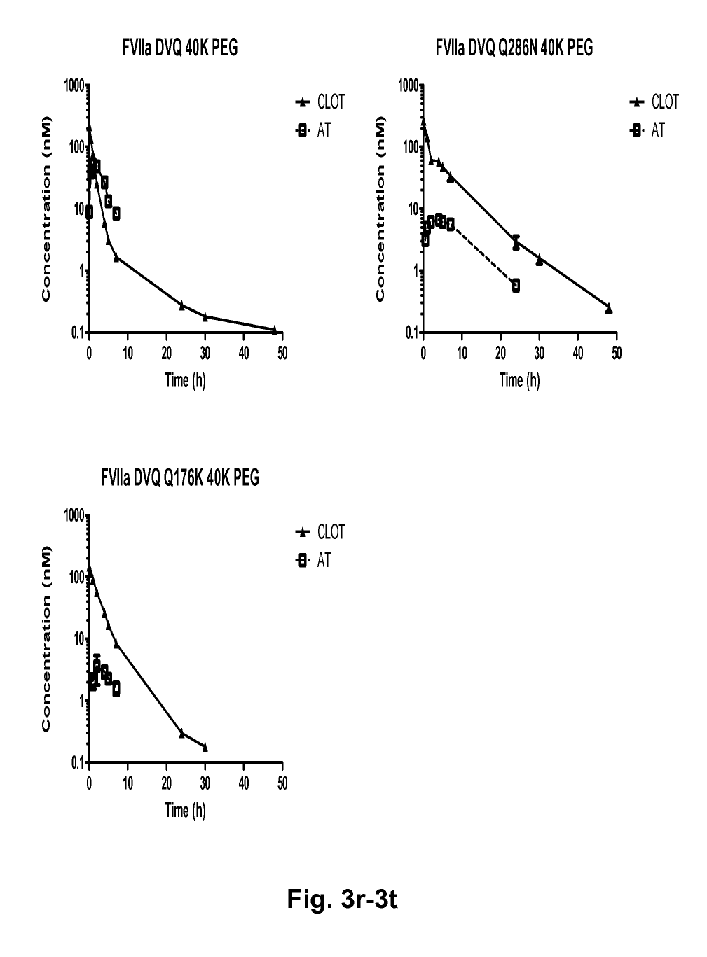

Potencies were estimated using a commercial FVIIa specific clotting assay; STACLOT®VIIa-rTF from Diagnostica Stago. The assay is based on the method published by J. H. Morrissey et al, Blood. 81:734-744 (1993). It measures sTF initiated FVIIa activity-dependent time to fibrin clot formation in FVII deficient plasma in the presence of phospholipids. Clotting times were measured on an ACL9000 (ILS) coagulation instrument and results calculated using linear regression on a bilogarithmic scale based on a FVIIa calibration curve. The same assay was used for measurements of FVIIa clotting activity in plasma samples from animal PK studies. The lower limit of quantification (LLOQ) in plasma was estimated to 0.25 U/ml. Plasma activity levels were converted to nM using the specific activity. Results are given in Table 2 and FIG. 3.

### Example 8

A selection of antithrombin resistant variants in unmodified and 40k-glycoPEGylated form were tested for their effect on thrombin generation and clot formation in FVIII-deficient plasma as described below. Results are given in Table 2.

Thrombin Generation Assay (TGA) of Human Donor Blood—

Thrombin generation was measured in platelet rich plasma (PRP) from healthy donors (final platelet concentration was 150×10 9/1). The PRP was treated with an inhibitory anti-human Factor VIII IgG to induce a haemophilia A-like condition. The platelets were activated by adding 100 μM final concentration of the PAR-1 agonist SFFLRN (Bachem, Bubendorf, Switzerland) and 100 ng/ml final concentration of the collagen receptor (GPVI) agonist convulxin (Pentapharm, Basel, Switzerland) approximately 5 minutes before initiating the assay. FVIIa and the FVIIa variants were added to microtiter plates in a volume of 20 μl together with 80 μl PRP containing platelet agonists. The reaction was initiated by adding 20 μl fluorogenic thrombin substrate containing CaCl₂ in a final concentration of 16.7 mM (FluCa Kit, Thrombinoscope by, Maastricht, The Netherlands). Thrombin generation was continuously measured for 120 minutes using a Fluoroscan Ascent® fluorometer (Thermo Fisher Scientific, Helsinki, Finland). The fluorescence signal was detected at wavelengths of 390 nm (excitation) and 460 nm (emission), corrected for α-2-macroglobulin-bound thrombin and converted to molar (nM) thrombin generated by means of a calibrator (Thrombinoscope) and Thrombinoscope software (Synapse BV, Maastricht, The Netherlands) as described (Hemker et al. 2003). The rate of thrombin generation was calculated as thrombin peak/(time to peak−lag time). Since no top plateau level could be obtained, EC₅₀ values could not be generated. Instead, the activity of variants relative to wild-type FVIIa was estimated by comparing the concentration of compound needed to obtain a certain rate represented on the steepest part on the graph.

Thromboelastography (TEG) of Human Whole Blood—

TEG analyses were performed in whole blood from healthy donors (essentially as described in Viuff et al Thrombosis Research, 2010; 126-144-149). The blood was treated with an inhibitory anti-human factor VIII IgG to induce a haemophilia A-like condition. FVIIa, FVIIa variants or buffer (HEPES 20 mM, NaCl 150 mM, BSA 2%) were added to tubes containing kaolin (Haemoscope, Niles, Ill., USA), and carefully mixed with whole blood by inversion of the tubes. The samples were transferred to TEG cups and re-calcified to initiate clotting. The haemostatic process was recorded by a TEG coagulation analyzer (5000 series TEG analyzer, Haemoscope Corporation, Chicago, USA). The TEG clotting time (R, denote the latency time from placing blood in the sample cup until the clot starts to form (2 mm amplitude), and the velocity of clot formation (MTG, maximum thrombus generation) were used for analysis. The samples were analyzed as single samples and the experiment performed twice (different donors each time). Data analysis was performed with Haemoscope Software version 4. EC₅₀ values were calculated based on a 4-parameter logistic concentration response curve fit for each parameter.

### Example 9

To explore the mechanism by which the identified substitutions affect antithrombin recognition, the crystal structure of a representative variant (FVIIa Q176K) was determined.

Purified H-D-Phe-Phe-Arg chloromethyl ketone (FFR-cmk; Bachem, Switzerland) active site inhibited FVIIa Q176K in complex with soluble Tissue Factor (fragment 1-209) was crystallized using the hanging drop method in accordance with (Bjelke et al. 2008). The protein buffer solution was a mix of 10 mM Tris-HCl, 100 mM NaCl, 15 mM CaCl₂, pH 7.5 and the protein concentration was 5.8 mg/ml. The precipitation, well, solution was: 100 mM sodium citrate, pH 5.6, 16.6% PEG 3350 and 12% 1-propanol. The hanging drops were set up in a 24-well VDX-plate, using a 1 ml of well solution, and with a mix of 1.5 μl of the protein solution and 0.5 μl of the well solution. The crystals grew as thin plates with dimensions up to 0.3×0.15×0.05 mm.

A crystal was transferred to a solution containing 80 vol. % crystallization well solution and 20% glycerol (99% purity). The crystal was let to soak for about 30 seconds after which the crystal was transferred to, and flash frozen in, liquid nitrogen. X-ray diffraction data were collected at beamline 911-3, the MAX-lab synchrotron, Lund, Sweden (Mammen et al., 2002). One part of the crystal was single, while other parts showed twinning. A complete data set from the un-twinned part of the crystal was obtained. The data were processed by the XDS data reduction software (Kabsch, 2010) resulting in a final resolution cut-off of 1.95 Å. Crystallographic data, refinement and model statistics are shown in Table 5.

The crystallographic coordinates of the 3ELA entry (Bjelke et al. 2008) from the Protein Data Bank (Berman et al., 2000), were used as starting model for rigid body refinements in REFMAC5 (Murshudov et al., 2011) of the CCP4 software package (Collaborative Computational Project, 1994). Refinements were followed by interactive model corrections in the computer graphics software COOT (Emsley et al., 2010). The coordinate refinements and model building were subsequently transferred to the PHENIX software package (Adams et al., 2010) and the PHENIX.REFINE software (Afonine et al., 2012). The final R- and R-free obtained were 0.183 and 0.216, respectively. Crystallographic data, refinement and model statistics are shown in Table 5.

TABLE 5
Crystallographic data, refinement and model statistics for the
FVIIa Q176K structure

| Space group | P 2₁ |
|---|---|
| Cell dimensions | 78.37, 69.37, 78.78, 90.46 |
| a, b, c [Å], β [°] | |
| Number of complex molecules in the | 1 |
| asymmetric unit | |
| Resolution | 29.5-1.95 (2.00-1.95) |
| Measured reflections | 229708 |
| Unique reflections | 61080 |
| ^{a}R_{sym} | 6.0 (77.4) |
| Completeness [%] | 98.9 (97.9) |
| I/σ(I) | 17.4 (1.9) |
| Wilson B factor [Å²] | 35.3 |
| Refinement cut-off | F > 0 σ F |
| Number of protein atoms | 4219 |
| Metal ions | 1 |
| Carbohydrate atoms | 21 |
| Inhibitor atoms | 34 |
| Glycerol atoms | 54 |
| Overall B factor value [Å²] | 48.3 |
| Number of TLS groups | 10 |
| ^{b}R_{cryst} | 18.3 (32.9) |
| ^{b}R_{free} | 21.6 (38.0) |
| Root mean square deviation | |
| Bonds | 0.018 |
| Angles | 1.69 |
| ^{d}Coordinate error [Å] | 0.27 |
| Ramachandran | |
| Favored regions | 496 (93.4%) |
| Allowed regions | 23 (4.36%) |
| Outlier region | 9 (1.70%) |

^{a}R_{sym} = ΣₕΣᵢ | I(h,i) − <I(h)> | / Σ IₕΣᵢ (h,i) where I(h,i) is the intensity of the ith measurement of h and <I(h)> is the corresponding average value of all i measurements.
^{b}R_{cryst} = Σₕ | |F(h)ₒ| − |F(h)_{c}| | / |F(h)ₒ|, where F(h)c is the calculated structure factor of reflection h, R_{free} is equivalent to R_{cryst}, but calculated for randomly chosen 5% of reflections that were omitted from the refinement process.
^{c}R-free is based on 5% of all reflections.
^{d}Maximun likelihood based

Structure Analysis—

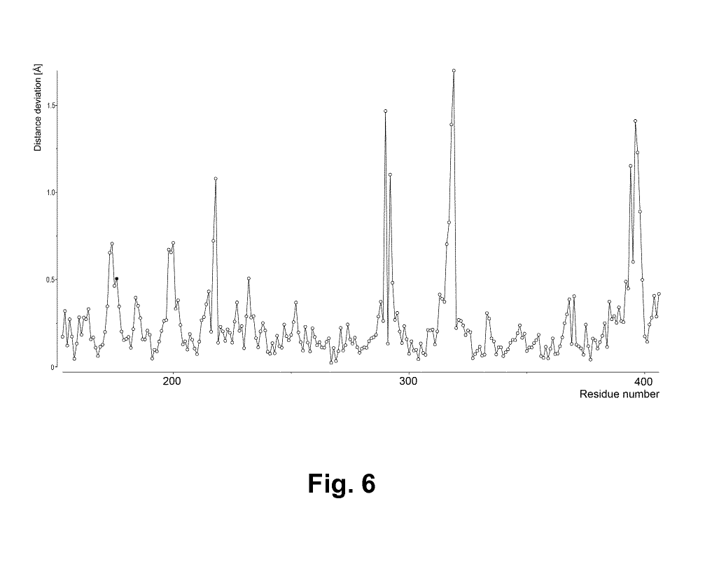

The heavy chain FVIIa Lys 176 residue is situated in the loop between beta-strands A1 and B1/in the very beginning of beta-strand B1. The electron density for the heavy chain Lys 176 residue is clearly shown for the main chain and until the side chain C-atom when using a 1.0 cut-off in a likelihood-weighted 2mFo-DFc map. The orientation of the Lys 176 side chain is in the direction of the heavy chain 293 Thr residue, some 3.5 Å away. A comparison of the FVIIIa Q176K structure with the Protein Data Bank (Berman, Westbrook, Feng, Gilliland, Bhat, Weissig, Shindyalov, & Bourne, 2000) 1 DAN structure (Banner et al., 1996) show a high similarity between the structures. The FVIIa FFR-cmk inhibited and Gla-domain containing 1 DAN structure crystallized in another space group, P4₁2₁2, and there is a small difference in inter-domain orientations. Root-mean-square deviations (RMSD) between Cα-atoms were calculated by GESAMT (Krissinel, 2012) and LSQKAB of the CCP4 software program package (Collaborative Computational Project, 1994). The overall RMSD for the three common chains, FVIIa heavy (H), FVIIa light (L) and Tissue Factor (T), of the two complexes is 0.796 Å (for 529 Cα-atoms pairs) while the RMSD for the FVIIa heavy chains only, the catalytic domain, is 0.347 Å. FIG. 6 show the individual Cα-Cα distances from a LSQKAB superimposition run between the catalytic domains of the FVIIa mutant Q176K and that from the 1 DAN structure.

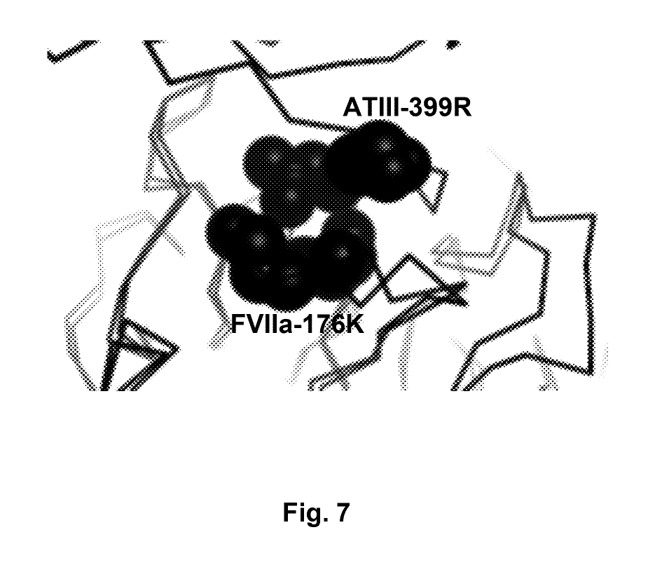

To study the possible interactions between antithrombin and the FVIIa mutant Q176K a superimposition of the FVIIa mutant was made on the Factor Xa molecule of the coordinates with the PDB-code 2GD4 (Johnson et al., 2006). The identity positions between the FXa and FVIIa molecule is 34.1% while the consensus positions are 49.4%. Superimposition using the GESAMT software gave and RMSD of 1.05 Å, Q=0.783 and an alignment length of 229 residues. From the riding antithrombin molecule it is then clear that if antithrombin interacts with FVIIa (Q176K) the two positively charged amino acids, Arg 399 of the antithrombin and K176 of the heavy chain of FVIIa will come very close (see Table 6) and FIG. 7), but, as both residues are positively charged the consequence is an electrostatic repulsion between the two residues. Moreover, the antithrombin Arg 399 is part of the reactive center loop (RCL) of the antithrombin molecule (Johnson et al. 2006) and a repulsion described will most probably negatively influence on the possibility for antithrombin to place its RCL into the active site of FVIIa. Thereby will a Q176K mutated FVIIa molecule be less susceptible to inhibition by antithrombin. This is in agreement with, and gives an explanation to, what is observed in Table 2, Table 3 and FIG. 3 showing increased resistance to inactivation by antithrombin, prolonged half-life and followed by only a slight decrease in activity.

TABLE 6
Distances between the two residues Q176K of FVIIa and Arg 399 of
complex (Johnson et al. 2006) has been superimposed and replaced
by the FVIIa mutant Q176K molecule.

| FVIIa Q176K | | | Antithrombin | | | |
|---|---|---|---|---|---|---|
| | Res. # | | | Res. # | | |
| Res. | and | Atom | Res. | and | Atom | Distance |
| Type | Chain | name | Type | Chain | name | [Å] |
| Lys | 176H | CB | Arg | 399I | NH1 | 3.79 |
| Lys | 176H | CE | Arg | 399I | NH1 | 3.31 |
| Lys | 176H | NZ | Arg | 399I | NH1 | 3.46 |
| Lys | 176H | O | Arg | 399I | CG | 3.82 |
| | | | Arg | 399I | CD | 3.04 |
| | | | Arg | 399I | NH1 | 3.90 |

### Example 10

In order to assess the effect of antithrombin resistance in a combination with activity enhancing substitutions and chemical modification, a selection of FVIIa variants (see Table 2) were conjugated to a 40-kDa PEG by enzymatic glyco-conjugation as described in the following section.

N-glycan directed PEGylation was carried out essentially as published elsewhere (Stennicke et al. 2008). Briefly, 4-aminobenzamidine (Sigma) was added to a final concentration of 10 mM to the protein (around 1.55 mg/ml) in solution in 10 mM Histidine, 50 mM NaCl, 10 mM CaCl₂, pH 5.8. A. Urifaciens sialidase was then added to a final concentration of 4 μg/ml to remove terminal sialic acids on the N-glycans. The desialylation reaction was carried out for 1 h at room temperature. The asialo-protein was subsequently purified by Gla domain-directed monoclonal antibody affinity chromatography as described elsewhere (Thim et al. 1988), using 50 mM Hepes, 100 mM NaCl, 10 mM CaCl₂, pH 7.4 to wash out the excess of benzamidine and 50 mM Hepes, 100 mM NaCl, 10 mM EDTA pH 7.4 as elution buffer. Calcium chloride and benzamidine were immediately added to the collected fractions to a final concentration of 10 mM.

The obtained product was analyzed by reducing- and non-reducing SDS-PAGE using 4-12% Bis-Tris Gels (Invitrogen) according to manufacturer's instructions. The protein concentration was determined by light-chain rpHPLC analysis. The obtained asialo-protein was homogenous based on both the SDS-PAGE and the RP-HPLC analysis. To the asialo-protein (final concentration around 26 μM) was added 40kDa-PEG-GSC (N-((2,3-di(20 kDa mPEGyl)propoxycarbonylamino)acetyl)-O²[5]cytidylyl-ξ-neuraminic acid; 10 equivalents) and ST3Gal-III (final concentration 0.22 U/ml). The reaction was carried out at 32° C. for 3 h. After capping of the glycoPEGylated product with NAN-CMP (cytidine-5′-monophosphate-N-acetyl neuraminic acid; 3 mM) for 1 h at 32° C., the product was isolated by antibody affinity chromatography as described above. The glycoPEGylated product was further purified by size-exclusion chromatography using a Superdex 200 pg 26/600 column (GE Healthcare). Fractions corresponding to the mono-glycoPEGylated product were pooled and analyzed by SDS-PAGE as described above. Subsequently the product was concentrated to around 1 mg/ml using an Amicon 10 kDa-cut off ultracentrifugation device (Millipore).

The content of di-glycoPEGylated FVIIa was assessed by analytical SEC HPLC using a TSK-Gel G300SW_{xL} column and detection by fluorescence (excitation 280 nm, emission at 354 nm) and absorbance (280 nm). The column temperature was 30° C. and the flow rate maintained at 1 ml/min in 200 mM Na-phosphate, 300 mM NaCl, 10% isopropanol, pH 6.9.

All variants tested (see list in Table 4) were amenable to glycoPEGylation and yielded a predominantly mono-PEGylated (>85%) product. In vitro characterization showed that the Q286N, Q176K and T293K variants in FVIIa wild-type, M298Q, and V158D/E296V/M298Q backgrounds exhibited a slight increase in antithrombin resistance upon glycoPEGylation. In addition, a lowering of the proteolytic activity was observed. However, Q176K and T293K-based variants still retained >100% TF-independent proteolytic activity in combination with M298Q and V158D/E296V/M298Q demonstrating the superiority of these mutations.

### Example 11

### Quantification Method

Hepylated FVIIa conjugates were analysed for purity by HPLC. HPLC was also used to quantify amount of isolated conjugate based on FVIIa reference molecules. Samples were analyzed either in non-reduced or reduced form. A Zorbax 300SB-C3 column (4.6×50 mm; 3.5 um Agilent, Cat. No.: 865973-909) was used. Column was operated at 30° C. 5 ug sample was injected, and column eluted with a water (A)-acetonitrile (B) solvent system containing 0.1% trifluoroacetic acid. The gradient program was as follows: 0 min (25% B); 4 min (25% B); 14 min (46% B); 35 min (52% B); 40 min (90% B); 40.1 min (25% B). Reduced samples were prepared by adding 10 ul TCEP/formic acid solution (70 mM tris(2-carboxyethyl)phosphine and 10% formic acid in water) to 25 ul/30 ug FVIIa (or conjugate). Reactions were left for 10 minutes at 70° C., before analysis on HPLC (5 ul injection).

### Preparation of HEP-Maleimide Polymers

Maleimide functionalized heparosan polymers of defined size are prepared by an enzymatic (PmHS1) polymerization reaction using the two sugar nucleotides UDP-GlcNAc and UDP-GlcUA. A priming trisaccharide (GlcUA-GlcNAc-GlcUA)NH₂ is used for initiating the reaction, and polymerization is run until depletion of sugar nucleotide building blocks. The terminal amine (originating from the primer) is then functionalized with suitable reactive group, in this case a maleimide functionality designed for conjugation to free cysteines. Reagents such as N-(g-maleimidobutyryloxy)sulfosuccinimide ester (sulfo-GMBS, Pierce) can be used for amine to maleimide conversion. Size of heparosan polymers can be pre-determined by variation in sugar nucleotide: primer stoichiometry. The technique is described in detail in US 2010/0036001.

### Selective Reduction of FVIIa Q286N 407C:

FVIIa Q286N 407C was reduced as described in US 20090041744 using a glutathione based redox buffer system. Non-reduced FVIIa Q286N 407C (20 mg) was incubated for 18h at 5° C. in a total volume of 18.2 ml 10 mM Hepes, 10 mM CaCl2, 50 mM NaCl, 0,01% Tween80, pH 6.0 containing 0.5 mM GSH, 15 uM GSSG, 25 mM p-aminobenzamidine and 2 μM Grx2. The solution was diluted with 20 ml 50 mM Hepes, 100 mM NaCl, pH 7.0 and cooled on ice. 4.0 ml 100 mM EDTA solution was then added while keeping pH neutral. The entire content was loaded onto two connected 5 ml HiTrap Q FF column (Amersham Biosciences, GE Healthcare) equilibrated in buffer A (50 mM HEPES, 100 mM NaCl, 1 mM EDTA, pH 7.0) to capture FVIIa Q286N 407C. After wash with buffer A to remove unbound glutathione buffer and Grx2p, FVIIa Q286N 407C was eluted in one step with buffer B (50 mM HEPES, 100 mM NaCl, 10 mM CaCl2, pH 7.0). The concentration of FVIIa Q286N 407C in the eluate was determined by HPLC. 17 mg FVIIa Q286N 407C was isolated in 12.2 ml 50 mM Hepes, 100 mM NaCl, 10 mM CaCl2, pH 7.0. When reaction was repeated a second time, a quantitative yield (20 mg) of 50 mM Hepes, 100 mM NaCl, 10 mM CaCl2, pH 7.0 was isolated in 8 ml 50 mM Hepes, 100 mM NaCl, 10 mM CaCl2, pH 7.0.

### Synthesis of 60k HEP-[C]-FVIIa Q286N 407C:

Single cysteine reduced FVIIa Q286N 407C (20 mg) was reacted with 60K HEP-maleimide (32 mg) in 50 mM Hepes, 100 mM NaCl, 10 mM CaCl2, pH 7.0 buffer (8.0 ml) for 14 hours at room temperature. The reaction mixture was then loaded on to a FVIIa specific affinity column (CV=25 ml) modified with a Gla-domain specific antibody and step eluted first with 2 column volumes of buffer A (50 mM Hepes, 100 mM NaCl, 10 mM CaCl2, pH 7.4) then two column volumes of buffer B (50 mM Hepes, 100 mM NaCl, 10 mM EDTA, pH 7.4). The method essentially follows the principle described by Thim, L et al. Biochemistry (1988) 27, 7785-779. The products with unfolded Gla-domain was collected and directly applied to two connected 5 ml HiTrap Q FF ion-exchange column (Amersham Biosciences, GE Healthcare, CV=10 ml) equilibrated with 10 mM His, 100 mM NaCl, pH 7.5. The column was washed with 4 column volumes of 10 mM His, 100 mM NaCl, pH 7.5 and 5 column volumes of 10 mM His, 100 mM NaCl, 10 mM CaCl2, pH=7.5 to eluted unmodified FVIIa Q286N 407C. The pH was then lowered to 6.0 with 10 mM His, 100 mM NaCl, 10 mM CaCl2, pH=6.0 (12 column volumes). 60k-HEP-[C]-FVIIa Q286N 407C was eluted with 10 column volumes of a 40% buffer A (10 mM His, 100 mM NaCl, 10 mM CaCl2, pH=6.0) and 60% buffer B (10 mM His, 1 M NaCl, 10 mM CaCl2, pH=6.0) solvent mixture. Fractions containing conjugate were combined, and dialyzed against 10 mM His, 100 mM NaCl, 10 mM CaCl2, pH=6.0 using a Slide-A-Lyzer cassette (Thermo Scientific) with a cut-off of 10kD. Yield (2.61 mg, 13%) was determined by quantifying the FVIIa light chain content against a FVIIa standard after tris(2-carboxyethyl)phosphine reduction using reverse phase HPLC. 12.6 mg un modified FVIIa Q286N 407C was also isolated.

### Selective Reduction of FVIIa T293K 407C:

FVIIa T293K 407C was reduced as described above for FVIIa Q286N 407C using a glutathione based redox buffer system. A total of 22.8 mg FVIIa T293K 407C was isolated in 12 ml 50 mM Hepes, 100 mM NaCl, 10 mM CaCl2, pH 7.0.

### Synthesis of 40k HEP-[C]-FVIIa T293K 407C:

Single cysteine reduced FVIIa T293K 407C (22 mg) and 40K HEP-maleimide (24 mg) was incubated for 18h at room temperature in 8 ml 50 mM Hepes, 100 mM NaCl, 10 mM CaCl2, pH 7.0 buffer containing 0.5 mM p-aminobenzamidine. The reaction mixture was then diluted with 20 ml 50 mM Hepes, 100 mM NaCl, 10 mM CaCl2, pH 7.4 and loaded on to a FVIIa specific affinity column (CV=64 ml) modified with a Gla-domain specific antibody. The column was step eluted first with two column volumes of buffer A (50 mM Hepes, 100 mM NaCl, 10 mM CaCl2, pH 7.4) then two column volumes of buffer B (50 mM Hepes, 100 mM NaCl, 10 mM EDTA, pH 7.4). The products with unfolded Gla-domain was collected and directly applied to three connected 5 ml HiTrap Q FF ion-exchange column (Amersham Biosciences, GE Healthcare, CV=15 ml) equilibrated with 10 mM His, 100 mM NaCl, pH 7.5. The column was washed with 4 column volumes of 10 mM His, 100 mM NaCl, pH 7.5 and 15 column volumes of 10 mM His, 100 mM NaCl, 10 mM CaCl2, pH=7.5 to eluted unmodified FVIIa T293K 407C. The pH was then lowered to 6.0 with 10 mM His, 100 mM NaCl, 10 mM CaCl2, pH=6.0 (12 column volumes). 40k-HEP-[C]-FVIIa T293K 407C was eluted with 15 column volumes of a 40% buffer A (10 mM His, 100 mM NaCl, 10 mM CaCl2, pH=6.0) and 60% buffer B (10 mM His, 1 M NaCl, 10 mM CaCl2, pH=6.0) solvent mixture. Fractions containing conjugate were combined, and dialyzed against 10 mM His, 100 mM NaCl, 10 mM CaCl2, pH=6.0 using a Slide-A-Lyzer cassette (Thermo Scientific) with a cut-off of 10 kD. Yield (15.3 mg, 69%) was determined by quantifying the FVIIa light chain content against a FVIIa standard after tris(2-carboxyethyl)phosphine reduction using reverse phase HPLC.

### Example 12

A pharmacokinetic analysis of the identified antithrombin resistance mutations alone or in combination with M298Q and V158D/E296V/M298Q and 40k-glycoPEGylation was performed in rats and dogs to assess their effect on the in vivo survival of FVIIa. Sprague Dawley rats (three per group) or Beagle dogs (two per group) were dosed intravenously. Stabylite™ (TriniLize Stabylite Tubes; Tcoag Ireland Ltd, Ireland) stabilized plasma samples were collected as full profiles at appropriate time points and frozen until further analysis. Plasma samples were analysed for clot activity (as described in Example 7) and by an ELISA quantifying FVIIa-antithrombin complexes. Pharmacokinetic analysis was carried out by non-compartmental methods using WinNonlin (Pharsight Corporation). The following parameters were estimated: Cmax (maximum concentration) of FVIIa-antithrombin complex, and T1/2 (the functional terminal half-life) and MRT (the functional mean residence time) for clot activity.

Briefly, FVIIa-antithrombin complexes were measured by use of an enzyme immunoassay (EIA). A monoclonal anti-FVIIa antibody that binds to the N-terminal of the EGF-domain and does not block antithrombin binding is used for capture of the complex (Dako Denmark A/S, Glostrup; product code 09572). A polyclonal anti-human AT antibody peroxidase conjugate was used for detection (Siemens Healthcare Diagnostics ApS, Ballerup/Denmark; product code OWMG15). A preformed purified complex of human wild-type or variant FVIIa and plasma-derived human antithrombin was used as standard to construct EIA calibration curves. Plasma samples were diluted and analysed and mean concentration of duplicate measurements calculated. The intra-assay precision of the EIA was between 1-8%.

Pharmacokinetic profiles are shown in FIG. 3 and estimated parameters listed Table 3. For the antithrombin resistant variants in FVIIa wild-type and 40k-glycoPEGylated background the accumulation of FVIIa-antithrombin complexes was reduced to below detection level. Furthermore, this was reflected in a significantly prolonged functional half-life of glyocPEGylated FVIIa Q286N (16 hrs in rat and 20 hrs in dog) compared to glycoPEGylated FVIIa (7.4 hrs in rat and 8 hrs in dog). Similarly in vivo inactivation by antithrombin was greatly diminished when the variants were further combined with M298Q and V158D/E296V/M298Q. Noticeably, the half-life of FVIIa M298Q T293K 40k-PEG was 13 hrs in rat compared to 7.4 hrs for FVIIa 40k-PEG, while retaining a proteolytic TF-independent activity of 222% (see Table 3).

### Example 13

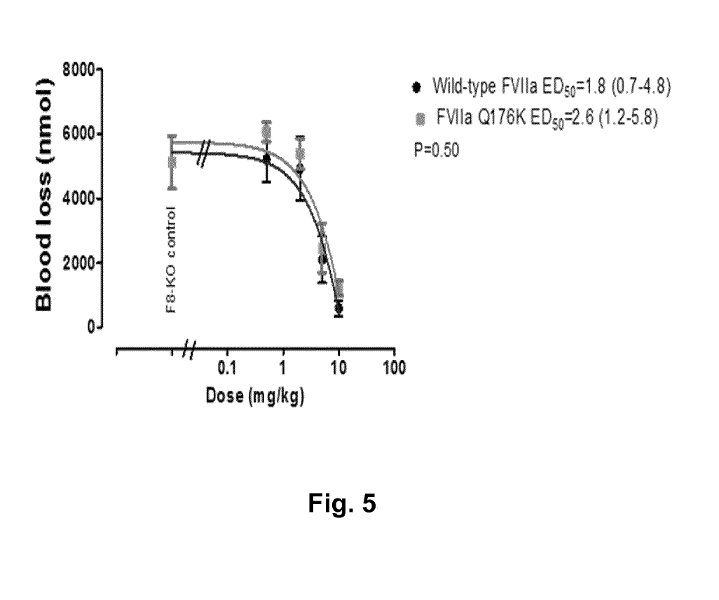

The in vivo efficacy of Q176K FVIIa compared to wild-type FVIIa was examined in the tail bleeding model in FVIII knock-out (Bi et al. 1996) mice at doses of 0.5, 2, 5 and 10 mg/kg. Tail bleeding was initiated in isofluran anesthetized mice by transection of 4 mm of the tip of the tail 5 min after dosing of FVIIa or vehicle intravenously in the tail vein. Bleeding time and blood loss was measured for a 30 minutes period in 37° C. saline as described elsewhere (Elm et al. 2012). Results are given in FIG. 5. The blood loss ED50 was calculated to 1.8 mg/kg for rFVIIa and 2.6 mg/kg for Q176K FVIIa, respectively, p=0.50. The bleeding time versus dose and the blood loss and bleeding time versus the exposure of wild-type FVIIa and FVIIa Q176K also show very similar dose response curves. In conclusions, there was no significant difference in dose response between the antithrombin resistant FVIIa Q176K variant and wild-type FVIIa in acute tail bleeding in FVIII deficient mice.

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., & Zwart, P. H. (2010). PHENIX: a comprehensive Python-based system for macromolecular structure solution. Acta Cryst. D 66, 213-221.
Afonine, P. V., Grosse-Kunstleve, R. W., Echols, N., Headd, J. J., Moriarty, N. W., Mustyakimov, M., Terwilliger, T. C., Urzhumtsev, A., Zwart, P. H., & Adams, P. D. (2012). Towards automated crystallographic structure refinement with phenix.refine. Acta Crystallogr. Sect. D-Biol. Crystallogr. 68, 352-367.
Banner, D. W., D'Arcy, A., Chéne, C., Winkler, F. K. Guha, A., Konigsberg, W. H., Nemerson, Y. and Kirchhofer, D. (1996) The crystal structure of the complex of blood coagulation factor VIIa with soluble tissue factor. Nature 380, 41-46.
Berman, H. M., Westbrook, J., Feng, Z., Gilliland, G., Bhat, T. N., Weissig, H., Shindyalov, I. N., & Bourne, P. E. (2000). The Protein Data Bank. Nucleic Acids Res. 28, 235-242.
Bi L., Sarkar R., Naas T., Lawler A. M., Pain J., Shumaker S. L. (2006) Further characterization of factor VIII-deficient mice created by gene targeting: RNA and protein studies. Blood 88, 3446-
Bjelke, J. R., Olsen, O. H., Fodje, M., Svensson, L. A., Bang, S., Bolt, G., Kragelund, B. B., & Persson, E. (2008). Mechanism of the Ca2+-induced enhancement of the intrinsic factor VIIa activity. J Biol Chem 283, 25863-25870.
Bock, P. E. (1992) Active-site-selective labeling of blood coagulation proteinases with fluorescence probes by the use of thioester peptide chloromethyl ketones. Specificity of thrombin labeling. J. Biol. Chem. 267, 14963-14973
Collaborative Computational Project, N. (1994). The CCP4 suite: programs for protein crystallography. Acta crystallographica. Section D, Biological crystallography 50, 760-763.
Elm T., Karpf D. M., Øvlisen K., Pelzer H., Ezban M., Kjalke M., Tranholm M. (2012) Pharmacokinetics and pharmacodynamics of a new recombinant FVIII (N8) in haemophilia A mice. Haemophilia 18, 139-145.
Emsley, P., Lohkamp, B., Scott, W. G., & Cowtan, K. (2010). Features and development of Coot. Acta Crystallogr. Sect. D-Biol. Crystallogr. 66, 486-501.
Freskgard, P. O., Olsen, O. H., and Persson, E. (1996) Structural changes in factor VIIa induced by Ca2+ and tissue factor studied using circular dichroism spectroscopy. Protein Sci. 5, 1531-1540
Hemker H. C., Giesen P., Al Dieri R., Regnault V., de Smedt E., Wagenvoord R., Lecompte T., Béguin S. (2003) Calibrated automated thrombin generation measurement in clotting plasma. Pathophysiol Haemost Thromb. 33, 4-15.
Higgins D G, Bleasby A J, and Fuchs R. (1992) CLUSTAL V: multiple alignment of DNA and protein sequences. Methods Mol Biol. 25, 307-318.
Johnson, D. J. D., Li, W., Adams, T. E. and Huntington, J. A. (2006) Antithrombin-S105A factor Xa-heparin structure reveals the allosteric mechanism of antithrombin activation. EMBO J. 25, 2029-2037.
Kabsch, W. (2010). XDS. Acta Crystallographica Section D Biological Crystallography 66, 125-132
Krissinel, E. (2012). Enhanced fold recognition using efficient short fragment clustering. Journal of Molecular Biochemistry 1, 76-85.
Mammen, C. B., Ursby, T., Cerenius, Y., Thunnissen, M., Als-Nielsen, J., Larsen, S., & Liljas, A. (2002). Design of a 5-station macromolecular crystallography beamline at MAX-Lab. Acta Phys. Pol. A 101, 595-602.
Murshudov, G. N., Skubak, P., Lebedev, A. A., Pannu, N. S., Steiner, R. A., Nicholls, R. A., Winn, M. D., Long, F., & Vagin, A. A. (2011). REFMAC5 for the refinement of macromolecular crystal structures. Acta Crystallographica Section D Biological Crystallography 67, 355-367.
Persson, E. and Nielsen, L. S. (1996) Site-directed mutagenesis but not gamma-carboxylation of Glu-35 in factor VIIa affects the association with tissue factor. FEBS Lett 385, 241-243
Stennicke, H. R., Ostergaard, H., Bayer, R. J., Kalo, M. S., Kinealy, K., Holm, P. K., Sorensen, B. B., Zopf, D., Bjorn, S. E. (2008) Generation and biochemical characterization of glycoPEGylated factor VIIa derivatives. Thromb Haemost. 100, 920-928
Thim, L., Bjoern, S., Christensen, M., Nicolaisen, E. M., Lund-Hansen, T., Pedersen, A. H., and Hedner, U. (1988) Amino acid sequence and posttranslational modifications of human factor VIIa from plasma and transfected baby hamster kidney cells. Biochemistry 27, 7785-779

The invention is further described by the following non-limiting embodiments:

### Embodiment 1

Factor VII(a) polypeptide comprising two or more substitutions relative to the amino acid sequence of human Factor VII (SEQ ID NO:1), wherein at least one of the substitutions is where T293 has been replaced by Lys (K), Tyr (Y), Arg (R) or Phe (F); where Q176 has been replaced by Lys (K), Arg (R), Asn (N); and/or Q286 has been replaced by Asn (N) and wherein at least one of the substitutions is where M298 has been replaced by Gln (Q), Lys (K), Arg (R), Asn (N). Gly (G), Pro (P), Ala (A), Val (V), Leu (L), Ile (I), Phe (F), Trp (W), Tyr (Y), Asp (D), Glu (E), His (H), Cys (C), Ser (S), or Thr (T).

### Embodiment 2

Factor VII(a) polypeptide according to embodiment 1, wherein T293 has been replaced by Lys (K), Tyr (Y), Arg (R) or Phe (F).

### Embodiment 3

Factor VII(a) polypeptide according to embodiment 1, wherein Q176 has been replaced by Lys (K), Arg (R), or Asn (N).

### Embodiment 4

Factor VII(a) polypeptide according to embodiment 1, wherein Q286 has been replaced by Asn (N)

### Embodiment 5

Factor VII(a) polypeptide according embodiments 1-4, wherein M298 has been replaced by Q.

### Embodiment 6

Factor VII(a) polypeptide according to embodiment 5, wherein as additional substitutions V158 has been replaced by Asp (D) and E296 has been replaced by Val (V).

### Embodiment 7

Factor VII(a) polypeptide according to embodiment 6, wherein as a further additional substitution K337 has been replaced by Ala (A).

### Embodiment 8

Factor VII(a) polypeptide according to embodiment 1, wherein the polypeptide has one of the following groups of substitutions T293K/M298Q, T293Y/M298Q, T293R/M298Q, T293F/M298Q, Q176K/M298Q, Q176R/M298Q, Q176N/M298Q, Q286N/M298Q, T293Y/V158D/E296V/M298Q, T293R/V158D/E296V/M298Q, T293K/V158D/E296V/M298Q, Q176K/V158D/E296V/M298Q or Q176R/V158D/E296V/M298Q.

### Embodiment 9

Factor VII(a) polypeptide according to embodiments 1-8, wherein the Factor VII(a) polypeptide is coupled with at least one half-life extending moiety.

### Embodiment 10

Factor VII(a) polypeptide according to embodiment 9, wherein the “half-life extending moiety” is selected from biocompatible fatty acids and derivatives thereof, Hydroxy Alkyl Starch (HAS) e.g. Hydroxy Ethyl Starch (HES), Poly Ethylen Glycol (PEG), Poly (Glyx-Sery)n (HAP), Hyaluronic acid (HA), Heparosan polymers (HEP), Phosphorylcholine-based polymers (PC polymer), Fleximers, Dextran, Poly-sialic acids (PSA), Fc domains, Transferrin, Albumin, Elastin like (ELP) peptides, XTEN polymers, PAS polymers, PA polymers, Albumin binding peptides, CTP peptides and FcRn binding peptides.

### Embodiment 11

Factor VII(a) polypeptide according to embodiment 10, wherein the half-life extending moiety is HEP.

### Embodiment 12

Factor VII(a) polypeptide according to any of embodiments 9-11, wherein the Factor VII(a) polypeptide has an additional mutation R396C, Q250C, or +407C.

### Embodiment 13

Factor VII(a) polypeptide according any of the preceding embodiments, wherein said Factor VII(a) polypeptide is disulfide linked to tissue factor.

### Embodiment 14

Factor VII(a) polypeptide according any of the preceding embodiments, wherein said Factor VII(a) polypeptide is a Factor VIIa variant with increased platelet affinity.

### Embodiment 15

Polynucleotide construct encoding a Factor VII(a) polypeptide according to any of embodiments 1-14.

### Embodiment 16

Host cell comprising the polynucleotide construct according to embodiment 15.

### Embodiment 17

Method for producing the Factor VII(a) polypeptide defined in any of embodiments 1-14.

### Embodiment 18

Pharmaceutical composition comprising a Factor VII(a) polypeptide as defined in any of embodiments 1-14.

### Embodiment 19

Pharmaceutical composition according to embodiment 18 for use as a medicament in the treatment of haemophilia A or B.

### Embodiment 20

Use of a Factor VII(a) polypeptide as defined in any of embodiments 1-14 for the preparation of a medicament for the treatment of bleeding disorders or bleeding episodes or for the enhancement of the normal haemostatic system.

### Embodiment 21

Use according to embodiment 20 for the treatment of haemophilia A or B.

### Embodiment 22

Method for the treatment of bleeding disorders or bleeding episodes in a subject or for the enhancement of the normal haemostatic system, the method comprising administering a therapeutically or prophylactically effective amount of a Factor VII(a) polypeptide as defined in any of embodiments 1-14 to a subject in need thereof.

### Embodiment 23

Factor VII(a) polypeptide as defined in any of embodiments 1-14 for use as a medicament.

### Embodiment 24

A Factor VII(a) polypeptide according to embodiment 23 for use as a medicament in the treatment of haemophilia A or B.

1. A Factor VII(a) polypeptide comprising two or more substitutions relative to the amino acid sequence of human Factor VII (SEQ ID NO:1),
wherein at least one of the substitutions is where T293 has been replaced by Lys (K), Tyr (Y), Arg (R) or Phe (F); where Q176 has been replaced by Lys (K), Arg (R), Asn (N); and/or Q286 has been replaced by Asn (N) and
wherein at least one of the substitutions is where M298 has been replaced by Gln (Q), Lys (K), Arg (R), Asn (N), Gly (G), Pro (P), Ala (A), Val (V), Leu (L), Ile (I), Phe (F), Trp (W), Tyr (Y), Asp (D), Glu (E), His (H), Cys (C), Ser (S), or Thr (T).

2. The Factor VII(a) polypeptide according to claim 1, wherein T293 has been replaced by Lys (K), Tyr (Y), Arg (R) or Phe (F).

3. The Factor VII(a) polypeptide according to claim 1, wherein Q176 has been replaced by Lys (K), Arg (R), or Asn (N).

4. The Factor VII(a) polypeptide according to claim 1, wherein Q286 has been replaced by Asn (N)

5. The Factor VII(a) polypeptide according to claim 1, wherein M298 has been replaced by Q.

6. The Factor VII(a) polypeptide according to claim 1, wherein the polypeptide has one of the following groups of substitutions T293K/M298Q, T293Y/M298Q, T293R/M298Q, T293F/M298Q, Q176K/M298Q, Q176R/M298Q, Q176N/M298Q, Q286N/M298Q, T293Y/V158D/E296V/M298Q, T293R/V158D/E296V/M298Q, T293K/V158D/E296V/M298Q, Q176K/V158D/E296V/M298Q or Q176R/V158D/E296V/M298Q.

7. The Factor VII(a) polypeptide according to claim 1, wherein the Factor VII(a) polypeptide is coupled with at least one half-life extending moiety.

8. The Factor VII(a) polypeptide according to claim 7, wherein the half-life extending moiety is selected from the group consisting of biocompatible fatty acids and derivatives thereof, Hydroxy Alkyl Starch (HAS), Hydroxy Ethyl Starch (HES), Poly Ethylen Glycol (PEG), Poly (Glyx-Sery)n (HAP), Hyaluronic acid (HA), Heparosan polymers (HEP), Phosphorylcholine-based polymers (PC polymer), Fleximers, Dextran, Poly-sialic acids (PSA), Fc domains, Transferrin, Albumin, Elastin like (ELP) peptides, XTEN polymers, PAS polymers, PA polymers, Albumin binding peptides, CTP peptides and FcRn binding peptides.

9. The Factor VII(a) polypeptide according to claim 8, wherein the half-life extending moiety is a heparosan polymer.

10. The Factor VII(a) polypeptide according to claim 7, wherein the Factor VII(a) polypeptide has an additional mutation R396C, Q250C, or 407C.

11. A method for producing the Factor VII(a) polypeptide according to claim 1.

12. A pharmaceutical composition comprising a Factor VII(a) polypeptide according to claim 1.

13. A method for treating a bleeding disorder or a bleeding episode in a subject or for enhancing normal haemostatic system, the method comprising administering a therapeutically or prophylactically effective amount of a Factor VII(a) polypeptide according to claim 1 to a subject in need thereof.

14. (canceled)

15. (canceled)

16. The method according to claim 13, wherein said bleeding disorder or bleeding episode is haemophilia A or B.
